SEQUENCE LISTING (15 sequences)

SEQ ID NO: 1 - protein, 7 aa, synthetic construct
  REGION 1..7: note = Description of Artificial Sequence: Binding protein
  VARIANT 5..6: note = Any amino acid
  VARIANT 7: note = L or I
RGDLXXX

SEQ ID NO: 2 - protein, 10 aa, synthetic construct
  REGION 1..10: note = Description of Artificial Sequence: Binding protein
  VARIANT 5..6: note = Any amino acid
  VARIANT 7: note = L or I
  VARIANT 10: note = R or K
RGDLXXXAQX

SEQ ID NO: 3 - protein, 20 aa, synthetic construct
  REGION 1..20: note = Description of Artificial Sequence: Binding protein
NAVPNLRGDLQVLAQRVART

SEQ ID NO: 4 - protein, 20 aa, Foot-and-mouth disease virus
NAVPNLRGDLQVLAQKVART

SEQ ID NO: 5 - protein, 21 aa, synthetic construct
  REGION 1..21: note = Description of Artificial Sequence: Binding protein
GNGVPNLRGDLQVLGQRVGRT

SEQ ID NO: 6 - protein, 10 aa, synthetic construct
  REGION 1..10: note = Description of Artificial Sequence: Binding protein
  VARIANT 5..6: note = Any amino acid
  VARIANT 8..10: note = Any amino acid
RGDLXXLXXX

SEQ ID NO: 7 - protein, 30 aa, synthetic construct
  REGION 1..30: note = Description of Artificial Sequence: Synthetic polypeptide
  VARIANT 5..6: note = Any amino acid
  VARIANT 8..10: note = Any amino acid
  REGION 11..30: note = Any helix promoting amino acid
  REGION 11..30: note = This region may encompass 1-20 residues
  REGION 1..30: note = See specification as filed for detailed description of substitutions and preferred embodiments
RGDLXXLXXXXXXXXXXXXXXXXXXXXXXX

SEQ ID NO: 8 - protein, 80 aa, synthetic construct
  REGION 1..80: note = Description of Artificial Sequence: Binding protein
  REGION 1..35: note = Any amino acid which enhances the hydrophobic interactions with the helix defined from LX1X2L and also enhances the RGD domain for binding
  SITE 1..35: note = This region may encompass 1-35 residues
  VARIANT 40..41: note = Any amino acid
  VARIANT 43..45: note = Any amino acid
  VARIANT 46..80: note = Any helix promoting amino acid
  REGION 46..80: note = This region may encompass 1-35 residues
  REGION 1..80: note = See specification as filed for detailed description of substitutions and preferred embodiments
XXXXXXXXXXXXXXXXXXXXXXXXXXXXXXXXXXXRGDLXXLXXXXXXXXXXXXXXXXXXXXXXXXXXXXXXXXXXXXXX

SEQ ID NO: 9 - protein, 20 aa, unidentified
  REGION 1..20: note = Description of Unknown: Latency-associated peptide of transforming growth factor beta sequence
GFTTGRRGDLATIHGMNRPF

SEQ ID NO: 10 - protein, 20 aa, Foot-and-mouth disease virus
YTASARGDLAHLTTTHARHL

SEQ ID NO: 11 - protein, 15 aa, synthetic construct
  REGION 1..15: note = Description of Artificial Sequence: Binding protein
  VARIANT 1..2: note = Any amino acid
  VARIANT 5..6: note = Any amino acid
  VARIANT 8: note = Any amino acid
  VARIANT 9: note = Any amino acid
  REGION 9..10: note = May or may not be present
  REGION 1..15: note = See specification as filed for detailed description of substitutions and preferred embodiments
XXDLXXLXXQKVART

SEQ ID NO: 12 - protein, 8 aa, unidentified
  REGION 1..8: note = Description of Unknown: AlphavBeta6-binding peptide sequence
RSDLTPLF

SEQ ID NO: 13 - protein, 9 aa, unidentified
  REGION 1..9: note = Description of Unknown: AlphavBeta6-binding peptide sequence
RSDLTPLFK

SEQ ID NO: 14 - protein, 8 aa, unidentified
  REGION 1..8: note = Description of Unknown: AlphavBeta6-binding peptide sequence
VGDLTYLK

SEQ ID NO: 15 - protein, 9 aa, unidentified
  REGION 1..9: note = Description of Unknown: AlphavBeta6-binding peptide sequence
VGDLTYLKK

(19) United States
(12) Patent Application Publication
(10) Pub. No.: US 2023/0183317 A1
(43) Pub. Date: Jun. 15, 2023

(54) METHODS OF DETECTING AND TREATING LUNG DAMAGE IN RESPIRATORY-RELATED VIRAL INFECTIONS

(71) Applicant: The Regents of the University of California, Oakland, CA (US)

(72) Inventor: Julie L. Sutcliffe, Davis, CA (US)

(73) Assignee: The Regents of the University of California, Oakland, CA (US)

(21) Appl. No.: 17/980,961

(22) Filed: Nov. 4, 2022

Related U.S. Application Data
(63) Continuation of application No. PCT/US2021/031248, filed on May 7, 2021.
(60) Provisional application No. 63/022,218, filed on May 8, 2020.

(51) Int. Cl. C07K 14/705; A61K 51/08; A61P 11/00; G01N 33/569; A61K 47/10

(52) U.S. Cl. CPC C07K 14/70546; A61K 51/082; A61P 11/00; G01N 33/56983; A61K 47/10

(57) ABSTRACT

Provided herein are methods for detecting, monitoring, diagnosing and treating respiratory-related viral infections, including SARS-CoV-2 infected individuals and subjects that are infected with or suspected of having been infected with the virus, or that have come in contact with COVID-19 suffering individuals.

### CROSS-REFERENCE TO RELATED APPLICATIONS

This application is a continuation of International Appl. No. PCT/US2021/031248, filed May 7, 2021, which claims priority to U.S. Provisional Application No. 63/022,218, filed May 8, 2020, the disclosures of which are herein incorporated by reference in their entirety for all purposes.

### BACKGROUND

At the end of 2019, Wuhan, Hubei Province, China experienced a large outbreak of a novel coronavirus infection. In humans, coronaviruses are among the spectrum of viruses that cause the common cold as well as more severe respiratory diseases-specifically, severe acute respiratory syndrome (SARS) and Middle East respiratory syndrome (MERS).

The novel coronavirus that infected Wuhan became known as severe acute respiratory syndrome coronavirus 2 (SARS-CoV-2) and the disease called by the virus, known as COVID-19. As of April 2020, cases of COVID-19 have been confirmed in countries throughout the world, and the spread of the disease designated as a pandemic.

There is a need for rapid and accurate detection and identification methods that can be used for detection, diagnosis, and management of patients that are infected with and/or are suspected of having been infected with SARS-CoV-2.

### BRIEF SUMMARY

Provided herein are methods that address the challenges of detecting, monitoring, diagnosing and treating respiratory-related viral infections, including SARS-CoV-2 infected individuals and subjects that are infected with or suspected of having been infected with the virus, or that have come in contact with a COVID-19 suffering individual.

CT images of patients infected with SARS-CoV-2 show a mix of consolidation and ground glass opacities in the lung. However, early identification was confounded by delayed radiographic presentations. There is increasing realization that significant biomarker and molecular imaging advancements will be essential to the improved diagnosis, prognosis, treatment and clinical study of lung damage following such infections. The αᵥβ₆ integrin is identified herein as one such promising biomarker; this integrin is an epithelial-specific receptor that is known to be upregulated in a variety of malignant tumors but also most applicable here, is upregulated in select injured tissues, including fibrotic lung.

In some aspects, provided herein is a method of imaging virus-related lung damage comprising: (a) administering to a subject a conjugate comprising an αᵥβ₆-binding peptide covalently attached to an imaging agent, wherein the subject has been exposed to or is suspected of exposure to a respiratory virus causative of lung damage; and (b) detecting the conjugate in lung tissue of the subject to determine the location and/or concentration of the conjugate in the lung tissue, thereby imaging the lung damage. In some embodiments, the lung damage comprises pulmonary fibrosis.

In some embodiments, the respiratory virus is a severe acute respiratory syndrome (SARS) coronavirus. In certain embodiments, the SARS coronavirus is selected from the group consisting of SARS-CoV, SARS-CoV-2, and variants thereof. In some embodiments, the subject has been diagnosed as a COVID-19 patient. In some embodiments, the subject has been previously quarantined for COVID-19 exposure. In some embodiments, the subject is symptomatic for COVID-19. In some embodiments, the subject is asymptomatic for COVID-19. In some embodiments, the subject is diagnosed as a COVID long-hauler.

In some embodiments, the subject has been infected with SARS-CoV-2 or a variant thereof. In some embodiments, the imaging is performed about 1 day, about 2 days, about 3 days, about 4 days, about 5 days, about 6 days, about 7 days, about 8 days, about 9 days, about 10 days, about 11, days, about 12 days, about 13 days, about 14 days, about 15 days, about 16 days, about 17 days, about 18 days, about 19 days, about 20 days, about 21 days, or more than about 21 days after infection with SARS-CoV-2 or a variant thereof.

In some embodiments, the subject has recovered from COVID-19. In certain instances, the subject has recovered from COVID-19 when the viral titre in the subject has reached an undetectable level. In other instances, the subject has recovered from COVID-19 when the subject tests negative for the presence of the virus. In some embodiments, the imaging is performed about 1 day, about 2 days, about 3 days, about 4 days, about 5 days, about 6 days, about 7 days, about 8 days, about 9 days, about 10 days, about 11, days, about 12 days, about 13 days, about 14 days, about 15 days, about 16 days, about 17 days, about 18 days, about 19 days, about 20 days, about 21 days, or more than about 21 days after the subject has recovered from COVID-19.

In some embodiments, the imaging is performed at a repeating interval. In some embodiments, the interval is about 1 week, about 2 weeks, about 3 weeks, about 4 weeks, about 5 weeks, about 6 weeks, about 7 weeks, about 8 weeks, about 9 weeks, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 2 years, about 3 years, about 4 years, about 5 years, or longer.

In some embodiments, steps (a) and (b) are repeated during the course of infection with the respiratory virus. In certain instances, steps (a) and (b) are repeated during the period where the subject tests positive for the virus. In other instances, steps (a) and (b) are repeated during the period where the subject experiences symptoms of the virus infection.

In some embodiments, steps (a) and (b) are repeated during the course of recovery from infection with the respiratory virus. In certain instances, steps (a) and (b) are repeated during the period where the subject shows an improvement in one or more symptoms of the virus infection. In other instances, steps (a) and (b) are repeated during the period where the subject has a decreasing viral titre.

In some embodiments, the imaging agent is selected from the group consisting of a radionuclide, biotin, a fluorophore, a fluorescent protein, an antibody, an enzyme, and combinations thereof. In certain instances, the imaging agent is a radionuclide selected from the group consisting of ¹¹C, ¹³N, ¹⁵O, ¹⁸F, ¹⁹F, ⁶¹Cu, ⁶²Cu, ⁶⁴Cu, ⁶⁷Cu, ⁶⁸Ga, ¹¹¹In, ¹²⁴I, ¹²⁵I, and ¹³¹I.

In some embodiments, the conjugate is detected by Magnetic Resonance Imaging (MRI), Magnetic Resonance Spectroscopy (MRS), Single Photon Emission Computerized Tomography (SPECT), Positron Emission Tomography (PET), optical imaging, or any combination of the aforementioned techniques. In some embodiments, the conjugate is administered to the subject by intravenous injection.

In some embodiments, step (b), the detecting step, is performed between about 10 minutes and about 180 minutes after step (a), the administering step. In some embodiments, step (b) is performed at about 15 minutes, about 30 minutes, about 60 minutes, about 75 minutes, about 90 minutes, or about 120 minutes after step (a). In some embodiments, the method includes repeating steps (a) and (b) for one or more additional time points and determining the extent of the regression or progression of lung damage over time. In some embodiments, the amount or concentration of the conjugate is correlative to the severity of lung damage caused by the respiratory virus.

In some embodiments, the conjugate further comprises a polyethylene glycol (PEG) moiety covalently attached to the amino-terminus of the peptide or a PEG moiety covalently attached to the C-terminus of the peptide. In certain embodiments, the imaging agent is covalently attached to the peptide or the PEG moiety. In some embodiments, the PEG moiety has a molecular weight of less than about 3000 daltons (Da). In certain instances, the PEG moiety is selected from the group consisting of PEG₁₂ (PEG 800), PEG₂₈ (PEG 1500), and (PEG₂₈)₂ (PEG 1500×2).

In some embodiments, the conjugate further comprises a first PEG moiety covalently attached to the amino-terminus of the peptide and a second PEG moiety covalently attached to the C-terminus of the peptide. In certain embodiments, the imaging agent is covalently attached to the peptide, the first PEG moiety, or the second PEG moiety. In some embodiments, the first PEG moiety and/or the second PEG moiety each have a molecular weight of less than about 3000 Da. In certain instances, the first PEG moiety and/or the second PEG moiety are independently selected from the group consisting of PEG₁₂ (PEG 800), PEG₂₈ (PEG 1500), and (PEG₂₈)₂ (PEG 1500×2).

In some embodiments, the αᵥβ₆-binding peptide comprises an RGD sequence. In certain embodiments, the αᵥβ₆-binding peptide comprises the amino acid sequence RGDLX₁X₂X₃, wherein X₁ and X₂ are independently selected amino acids and X₃ is L or I. In certain embodiments, the αᵥβ₆-binding peptide comprises the amino acid sequence RGDLX₁X₂X₃AQX₆, wherein X₆ is R or K. In particular embodiments, the αᵥβ₆-binding peptide comprises an amino acid sequence selected from the group consisting of NAVPNLRGDLQVLAQRVART (A20FMDV2 K16R), NAVPNLRGDLQVLAQKVART (A20FMDV2), and GNGVPNLRGDLQVLGQRVGRT.

In some embodiments, the conjugate comprises the structure:

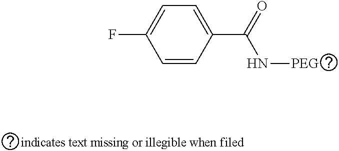

In some aspects, provided herein is a method of treating virus-related lung damage comprising: administering to a subject a conjugate comprising an αᵥβ₆-binding peptide covalently attached to a therapeutic agent, wherein the subject has been exposed, is diagnosed with or suffers from a respiratory virus causative of lung damage, thereby treating the lung damage. In some embodiments, the lung damage comprises pulmonary fibrosis. In some embodiments, the conjugate is administered to the subject by intravenous injection.

In some embodiments, the respiratory virus is a severe acute respiratory syndrome (SARS) coronavirus. In certain embodiments, the SARS coronavirus is selected from the group consisting of SARS-CoV, SARS-CoV-2, and variants thereof. In some embodiments, the subject has been diagnosed as a COVID-19 patient. In some embodiments, the subject has been infected with SARS-CoV-2 or a variant thereof. In some embodiments, the subject is symptomatic for COVID-19. In some embodiments, the subject is diagnosed as a COVID long-hauler.

In some embodiments, the therapeutic agent is an anti-inflammatory, an anti-fibrotic, or an antiviral agent. In certain embodiments, the therapeutic agent is a small molecule. In particular embodiments, the therapeutic agent is selected from the group consisting of nintedanib, pirfenidone, remdesivir, and hydroxychloroquine. In some embodiments, the therapeutic agent is covalently attached to the peptide via a linker. In certain instances, the linker is a cleavable linker.

In some embodiments, the conjugate further comprises a PEG moiety covalently attached to the amino-terminus of the peptide or a PEG moiety covalently attached to the C-terminus of the peptide. In certain embodiments, the therapeutic agent is covalently attached to the peptide or the PEG moiety. In some embodiments, the PEG moiety has a molecular weight of less than about 3000 Da. In certain instances, the PEG moiety is selected from the group consisting of PEG₁₂ (PEG 800), PEG₂₈ (PEG 1500), and (PEG₂₈)₂ (PEG 1500×2).

In some embodiments, the conjugate further comprises a first PEG moiety covalently attached to the amino-terminus of the peptide and a second PEG moiety covalently attached to the C-terminus of the peptide. In certain embodiments, the therapeutic agent is covalently attached to the peptide, the first PEG moiety, or the second PEG moiety. In some embodiments, the first PEG moiety and/or the second PEG moiety each have a molecular weight of less than about 3000 Da. In certain instances, the first PEG moiety and/or the second PEG moiety are independently selected from the group consisting of PEG₁₂ (PEG 800), PEG₂₈ (PEG 1500), and (PEG₂₈)₂ (PEG 1500×2).

In some embodiments, the αᵥβ₆-binding peptide comprises an RGD sequence. In certain embodiments, the αᵥβ₆-binding peptide comprises the amino acid sequence RGDLX₁X₂X₃, wherein X₁ and X₂ are independently selected amino acids and X₃ is L or I. In certain embodiments, the αᵥβ₆-binding peptide comprises the amino acid sequence RGDLX₁X₂X₃AQX₆, wherein X₆ is R or K. In particular embodiments, the αᵥβ₆-binding peptide comprises an amino acid sequence selected from the group consisting of NAVPNLRGDLQVLAQRVART (A20FMDV2 K16R), NAVPNLRGDLQVLAQKVART (A20FMDV2), and GNGVPNLRGDLQVLGQRVGRT.

Other objects, features, and advantages of the present invention will be apparent to one of skill in the art from the following detailed description and figures.

### BRIEF DESCRIPTION OF THE DRAWINGS

FIG. 1 shows axial CT (left) and PET/CT (right) images 1 hour after 10 mCi injection of ¹⁸F-αᵥβ₆-BP; ground glass opacity noted in right upper lobe of the lung.

FIG. 2 shows an initial chest X-ray at hospital admission showing diffuse pulmonary opacities in the mid and peripheral lungs bilaterally, consistent with diagnosis of SARS-CoV-2 associated pneumonia.

FIG. 3 shows a chest CT scan on day 4 after admission. Transaxial view of the upper lung (left) and lower lung (right) demonstrating moderate to severe bilateral central and peripheral patchy areas of ground glass and consolidative changes throughout the lungs (arrows).

FIG. 4 shows transaxial CT (left), attenuation-corrected ¹⁸F-αᵥβ₆-BP PET (middle; scale: SUVmax of 5), and ¹⁸F-αᵥβ₆-BP PET/CT (right) images through upper lungs (top) and lower lungs (bottom), showing increased uptake and areas of bilateral patchy opacity. Transaxial CT images through the upper lungs (top left) and lower lung (bottom left) demonstrating improved areas of bilateral patchy opacities (arrows) at time of the ¹⁸F-αᵥβ₆-BP PET/CT scan. Transaxial co-registered attenuation-corrected ¹⁸F-αᵥβ₆-BP PET images (scale: SUVmax of 5) through the upper lungs (middle panel top) and lower lung (middle panel bottom) demonstrating elevated activity (SUVmax of 3, arrows) in areas corresponding to areas of opacities on the CT. Regions of normal lung parenchyma demonstrated SUVmax of 0.8-1.0.

FIG. 5 shows three imaging time points each taken 3 months apart. ¹⁸F-αᵥβ₆-BP PET scan (right panel in each scan): Elevated activity on PET concordant with abnormalities seen on associated CT with an approximate 3:1 ratio (activity to background lung) on scans 1 and 2 in the upper lobes and reducing to 2:1 on the third scan. The lower lobes on scans 2 and 3 have returned to close to background lung activity. CT (left panel in each scan): Progressive improvement in ground glass and airspace opacities in the upper and to a greater degree the lower lobes of the lungs with resulting small amount of linear arcades vs scarring in the lower lungs on the final scan.

FIG. 6 shows the structure of PEG₂₈-[A20FMDV2(K16R)]-PEG₂₈, ¹⁸F-αᵥβ₆-BP.

FIG. 7 shows a synthetic route for a conjugate of αᵥβ₆-BP and a therapeutic agent.

### DETAILED DESCRIPTION

Respiratory viruses such as SARS, including SARS-CoV-2, are implicated in lung damage in infected patients. Such lung damage includes lung fibrosis, such as lung fibrosis associated with pneumonia and other lung-related complications of SARS.

The accurate diagnosis of lung damage can assist in the early diagnosis of subjects having COVID-19 and play a role in managing the disease, even where there are as of yet no proven or available therapies for the treatment of COVID-19. Chest x-ray (CT) has been utilized for such diagnosis. However, there are limits of detection for CT that may limit how early in infection CT may provide the requires sensitivity for early diagnosis. Similarly, such limitations of CT may limit its applicability in following patients as they progress through the course of COVID-19 and/or the course of recovery.

The compositions and methods provided herein provide a sensitive and targeted imaging of lung damage, particularly lung fibrosis, caused by or induced by infection with SARS-CoV-2. Provided herein are compositions and methods for detecting lung damage in subjects having or suspected of having a respiratory-related viral infection, such as severe acute respiratory syndrome (SARS) coronavirus, for example SARS-CoV and SARS-CoV-2. The methods herein utilize an integrin-binding peptide directed to binding integrin αᵥβ₆ to enable in vivo detection of the integrin αᵥβ₆ with imaging systems such as PET. The αᵥβ₆-binding peptide (αᵥβ₆-BP) is rapidly internalized into receptor-positive cells and this selective internalization enables visualization of receptor-positive cells when the αᵥβ₆-BP is linked to an imaging agent.

Also provided herein are compositions and methods for treating a subject infected with SARS-CoV-2. The methods herein utilize an integrin-binding peptide directed to binding integrin αᵥβ₆ on an infected cell to deliver a conjugated drug to the infected cell. In some embodiments, the conjugate includes a cleavable linker between the integrin-binding peptide and the therapeutic agent, such that the therapeutic agent is released intracellularly once delivered to the targeted infected cell. For example, the conjugate includes a cathepsin cleavable linker. The αᵥβ₆-BP is rapidly internalized into receptor-positive cells and this selective internalization enables delivery of the therapeutic agent to receptor-positive cells when the αᵥβ₆-BP is linked to the therapeutic agent.

### Conjugates

In some aspects, the αᵥβ₆-BP is a conjugate that includes the αᵥβ₆-BP and an imaging agent. In some aspects, the αᵥβ₆-BP is a conjugate that includes the αᵥβ₆-BP and a therapeutic agent. In some embodiments, the conjugate further includes a first polyethylene glycol (PEG) moiety covalently attached to the amino-terminus of the αᵥβ₆-BP and a second PEG moiety covalently attached to the carboxyl-terminus of the αᵥβ₆-BP. In some embodiments, the imaging agent or therapeutic agent is attached to the αᵥβ₆-BP, the first PEG moiety, or the second PEG moiety. In certain embodiments, the imaging agent or therapeutic agent is covalently attached as the most N-terminal moiety in the conjugate. In some embodiments, the therapeutic agent is covalently attached with a cleavable linker, such as a cathepsin-cleavable linker, to the αᵥβ₆-BP, the first PEG moiety, or the second PEG moiety.

In some embodiments, the first PEG moiety and the second PEG moiety each have a molecular weight of less than about 5000 daltons (Da). In particular embodiments, the first PEG moiety and the second PEG moiety each have a molecular weight of less than about 3000 Da. In certain embodiments, the first PEG moiety and the second PEG moiety are monodisperse PEG moieties having a defined chain length. PEG moieties having a defined chain length generally include PEG molecules of discrete molecular weights with an exactly defined number of repeating ethylene glycol units. Non-limiting examples of PEG moieties having a defined chain length include small, monodisperse PEG molecules having greater than about 90%, 910%, 92%, 93%, 94%, or 95% oligomer purity.

In certain instances, the first PEG moiety and the second PEG moiety are independently selected from the group consisting of PEG₁₁, PEG₁₂ (PEG 800), PEG₂₈ (PEG 1500), and (PEG₂₈)₂ (PEG 1500×2). In certain embodiments, the first PEG moiety and the second PEG moiety are the same. In particular embodiments, the first PEG moiety and the second PEG moiety are both PEG₂₈ (PEG 1500). Other non-limiting examples of PEG units suitable for use as the first and/or second PEG moiety with the conjugates herein include PEG 200, PEG 300, PEG 400, PEG 500, PEG 600, PEG 700, PEG 900, PEG 1000, PEG 1100, PEG 1200, PEG 1300, PEG 1400, PEG 1600, PEG 1700, PEG 1800, PEG 1900, PEG 2000, PEG 2100, PEG 2200, PEG 2300, PEG 2400, PEG 2500, PEG 2600, PEG 2700, PEG 2800, PEG 2900, PEG 3000, PEG 3250, PEG 3350, PEG 3500, PEG 3750, PEG 4000, PEG 4250, PEG 4500, PEG 4750, and PEG 5000, as well as derivatives thereof such as branched PEG derivatives. In particular embodiments, these PEG molecules contain an exactly defined number of repeating units “n” and are monodisperse (e.g., having greater than about 95% oligomer purity). PEG moieties suitable for use are commercially available from EMD Chemicals, Inc. (San Diego, Calif.) and Polypure AS (Oslo, Norway).

In some embodiments, the conjugates described herein include an imaging agent covalently attached to the αᵥβ₆-BP, the first PEG moiety, and/or the second PEG moiety. In particular embodiments, the imaging agent is covalently attached to the first PEG moiety, i.e., at the amino-terminal end of the αᵥβ₆-BP. In some embodiments, the imaging agent is selected from the group consisting of a radionuclide, biotin, a fluorophore, a fluorescent protein, an antibody, an enzyme such as horseradish peroxidase or alkaline phosphatase, and combinations thereof. In certain embodiments, the radionuclide is selected from the group consisting of ¹¹C, ¹³N, ¹⁵O, ¹⁸F, ¹⁹F, ⁶¹Cu, ⁶²Cu, ⁶⁴Cu, ⁶⁷Cu, ⁶⁸Ga, ¹¹¹In, ¹²⁴I, ¹²⁵I, and ¹³¹I.

In some embodiments, the radionuclide is attached via a prosthetic group to the αᵥβ₆-BP, the first PEG moiety, or the second PEG moiety. In certain embodiments, the radionuclide is attached via a prosthetic group to the first PEG moiety. In particular embodiments, the radionuclide is attached via a prosthetic group as the most N-terminal moiety in the conjugate. Non-limiting examples of prosthetic groups include benzoyl groups (e.g., fluorobenzoic acid (FBA)), fluoropropionic acid (FPA), pyridine (Py), dipyridyl-tetrazine (Tz), trans-cyclooctene (TCO), derivatives thereof, and combinations thereof. In some embodiments, the radionuclide is ¹⁸F or ¹⁹F covalently attached to the first PEG moiety via a benzoyl group such as FBA. For example, 4-[¹⁸F]-fluorobenzoic acid ([¹⁸F]FBA) or 4-[¹⁹F]-fluorobenzoic acid ([¹⁹F]FBA) can be used to radiolabel the conjugates.

In some embodiments, the radionuclide is attached via a chelating agent to the αᵥβ₆-BP, the first PEG moiety, or the second PEG moiety. In certain embodiments, the radionuclide is attached via chelating agent to the first PEG moiety. In particular embodiments, the radionuclide is attached via a chelating agent as the most N-terminal moiety in the conjugate.

In some embodiments, the conjugates described herein include a therapeutic agent covalently attached to the αᵥβ₆-BP, the first PEG moiety, and/or the second PEG moiety. In certain embodiments, the therapeutic agent is a small molecule. In particular embodiments, the therapeutic agent is an anti-viral agent, an anti-inflammatory agent or an anti-fibrotic agent. Non-limiting examples of therapeutic agents include nintedanib, pirfenidone, remdesivir, hydroxychloroquine, dexamethasone, prednisone, and methylprednisolone.

In some embodiments, the conjugate of an imaging agent or therapeutic agent further comprises an albumin binding motif covalently attached to the αᵥβ₆-BP, the first PEG moiety, or the second PEG moiety. In particular embodiments, the albumin binding motif is 4-(4-iodophenyl)butyric acid (IPA) or a homolog thereof with a shorter alkyl chain such as, e.g., 4-(4-iodophenyl)propionic acid or 4-(4-iodophenyl)acetic acid. In certain instances, the albumin binding motif is covalently attached to the first and/or second PEG moiety via a linker such as a glutamic acid (E) linker or other suitable linker (e.g., amino acid or peptide linker) known to one of skill in the art. In certain embodiments, the albumin binding motif is ε-(4-(4-iodophenyl)butyl amide)lysine-glutamic acid (“K(IPA)E”), which corresponds to IPA that is covalently attached to the side-chain of the lysine residue of a lysine-glutamic acid peptide linker. In some embodiments, the K(IPA)E albumin binding motif is covalently attached to the first PEG moiety. In other embodiments, the imaging agent is covalently attached (e.g., via a prosthetic group, a chelating agent, or a linker) to an albumin binding motif that is covalently attached to the first PEG moiety.

αᵥβ₆ Integrin-Binding Peptides of the Conjugates

In some embodiments, the αᵥβ₆ integrin-binding peptide (also referred to herein as αᵥβ₆-BP) comprises the amino acid sequence RGDLX₁X₂X₃, wherein X₁ and X₂ are independently selected amino acids and X₃ is L or I. In some instances, X₁ and X₂ are independently selected from the group consisting of Glu, Ala, Leu, Met, Gln, Lys, Arg, Val, Ile, His, Thr, Trp, Phe, and Asp. In certain embodiments, X₁ is Q, X₂ is V, and X₃ is L. In particular embodiments, the αᵥβ₆-BP comprises the amino acid sequence RGDLX₁X₂X₃AQX₆, wherein X₆ is K or R. In certain instances, X₆ is R.

In some embodiments, the residues LX₁X₂X₃ are present within an α-helix. An α-helix is understood to be a sequential group of amino acids in a peptide that interact with a particular hydrogen bonding pattern and thus define a helical structure. For example, the hydrogen bonding pattern in a standard α-helix is between the carbonyl oxygen of residue n and the amide hydrogen of residue n+4. For a 3₁₀-helix, this hydrogen bonding pattern is between residues n and n+3. For a pi-helix, this hydrogen bonding pattern is between residues n and n+5. The number of residues per turn in each α-helix is 3.6, 3.0, and 4.4 for the standard α-helix, 3₁₀-helix, and pi-helix, respectively. In one embodiment, the α-helix of the αᵥβ₆-BP enables the hydrophobic side-chains of the residues LX₁X₂L/I to protrude from one side of the helix. In another embodiment, the α-helix has at least one turn. An α-helix may be an α-helix mimetic as described in, e.g., International Publication No. WO 95/00534. α-helix mimetics are α-helical structures which are able to stabilize the structure of a naturally-occurring or synthetic peptide.

The αᵥβ₆-BP used in the conjugates described herein may comprise standard helices, 3₁₀-helices, pi-helices, or any combination thereof. For example, the helices may comprise amino acids that form a “cap” structure, such as an amino-terminal cap and/or a carboxyl-terminal cap which flank the helix.

In other embodiments, the αᵥβ₆-BP comprises the sequence RGDLX₁X₂LX₄X₅X₆, wherein X₁, X₂, X₄, X₅, and X₆ are independently selected amino acids. In certain instances, X₁, X₂, X₄, X₅, and X₆ are helix-promoting residues. For example, the helix-promoting residues can be independently selected from the group consisting of Glu, Ala, Leu, Met, Gln, Lys, Arg, Val, Ile, His, Thr, Trp, Phe, and Asp. The helix-promoting residues can comprise naturally-occurring amino acids or unnatural amino acids such as artificial or modified amino acids. In some embodiments, the αᵥβ₆-BP comprises the sequence RGDLX₁X₂LX₄X₅X₆Zn, wherein Z is a helix-promoting residue and n is any number between 1 and 20. In particular embodiments, n is between 5 and 15 or between 8 and 12. Extension of the helix to include helical residues in the Z position can further increase the helix dipole and provide enhanced binding to αᵥβ₆ integrin.

In further embodiments, the αᵥβ₆-BP may be represented by the formula: BmRGDLX₁X₂LX₄X₅X₆Zn, wherein B is m amino acids which enhances the hydrophobic interactions with the helix defined from LX₁X₂L and also enhances the RGD domain for binding, Z is a helix-promoting residue, n is a number between 1 and 35, and m is a number between 1 and 35. In particular embodiments, m is selected so that B is sufficiently long to facilitate a hydrophobic/non-covalent interacting core. The exact nature of these residues depends on the general design of the region. In particular, it is preferred to have a mixture of hydrophobic interactions (from residues such as Val, Ile, Leu) and/or electrostatic interactions (using Asp, Glu, Lys, and/or Arg together with their counterpart ion-pair at X₁ and/or X₂).

In certain embodiments, the αᵥβ₆-BP comprises the amino acid sequence RGDLX₁X₂X₃AQX₆, wherein X₆ is Lys (K) or Arg (R). In particular embodiments, X₆ is R. In some embodiments, the αᵥβ₆-BP comprises or consists of an amino acid sequence selected from the group consisting of NAVPNLRGDLQVLAQKVART (A20FMDV2), NAVPNLRGDLQVLAQRVART (A20FMDV2 K16R), GNGVPNLRGDLQVLGQRVGRT, GFTTGRRGDLATIHGMNRPF (A20LAP), YTASARGDLAHLTTTHARHL (A20FMDV1), and combinations thereof. In particular embodiments, the αᵥβ₆-BP comprises or consists of an amino acid sequence selected from the group consisting of NAVPNLRGDLQVLAQKVART (A20FMDV2), NAVPNLRGDLQVLAQRVART (A20FMDV2 K16R), and GNGVPNLRGDLQVLGQRVGRT.

In some embodiments, the αᵥβ₆-BP comprises the amino acid sequence X₁X₂DLX₃X₄LX₅(X₆)ₘ(Q)ₙKVART, wherein where m and n are independently 0 or 1; and X₁, X₂, X₃, X₄, X₅, and X₆ are independently selected amino acids, provided that X₃ is not Q when X₄ is V. In some embodiments, the αᵥβ₆-BP comprises the amino acid sequence RSD or VGD, e.g., the αᵥβ₆-BP comprises the sequence RSDLTPLF, RSDLTPLFK, VGDLTYLK, VGDLTYLKK, or any of the αᵥβ₆-BP sequences disclosed in International Publication Nos. WO 2015/160770, WO 2017/218569, and WO 2020/051549, which are incorporated herein by reference in their entirety.

In some embodiments, the αᵥβ₆-BP of the conjugates described herein is between about 8 and about 45 amino acids in length. In certain instances, the αᵥβ₆-BP is 20 amino acids in length. In other embodiments, the αᵥβ₆-BP is between about 5 to about 45 amino acids in length, between about 8 to about 45 amino acids in length, between about 8 to about 25 amino acids in length, between about 12 to about 45 amino acids in length, between about 5 to about 40 amino acids in length, between about 10 to about 40 amino acids in length, or about 35, 30, 25, 20, 15, or 10 amino acids in length. For example, the αᵥβ₆-BP may be about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or more amino acids in length. In particular embodiments, the αᵥβ₆-BP is about 21 or more amino acids in length.

The αᵥβ₆-BP used in the conjugates described herein can also be functional variants of the peptides as defined above, including peptides that possess at least about 50%, 55%, 60%, 65%, 70%, 75%, 80%, 85%, 90%, 95%, or more sequence identity with the peptides described above. In certain instances, the αᵥβ₆-BP can comprise naturally-occurring amino acids and/or unnatural amino acids. Examples of unnatural amino acids include, but are not limited to, D-amino acids, ornithine, diaminobutyric acid ornithine, norleucine ornithine, pyriylalanine, thienylalanine, naphthylalanine, phenylglycine, alpha and alpha-disubstituted amino acids, N-alkyl amino acids, lactic acid, halide derivatives of naturally-occurring amino acids (e.g., trifluorotyrosine, p-Cl-phenylalanine, p-Br-phenylalanine, p-I-phenylalanine, etc.), L-allylglycine, b-alanine, L-a-amino butyric acid, L-g-amino butyric acid, L-a-amino isobutyric acid, L-e-amino caproic acid, 7-amino heptanoic acid, L methionine sulfone, L-norleucine, L-norvaline, p-nitro-L-phenylalanine, L-hydroxyproline, L-thioproline, methyl derivatives of phenylalanine (e.g., 1-methyl-Phe, pentamethyl-Phe, L-Phe (4-amino), L-Tyr (methyl), L-Phe(4-isopropyl), L-Tic (1,2,3,4-tetrahydroisoquinoline-3-carboxyl acid), L-diaminopropionic acid, L-Phe (4-benzyl), etc.). The αᵥβ₆-BP may be further modified. For example, one or more amide bonds may be replaced by ester or alkyl backbone bonds. There may be N- or C-alkyl substituents, side-chain modifications, or constraints such as disulfide bridges or side-chain amide or ester linkages.

The αᵥβ₆-BP used in the conjugates described herein may include both modified peptides and synthetic peptide analogues. The αᵥβ₆-BP may be modified to improve formulation and storage properties, or to protect labile peptide bonds by incorporating non-peptidic structures. The αᵥβ₆-BP may be prepared using methods known in the art. For example, the αᵥβ₆-BP may be produced by chemical synthesis, e.g., using solid phase techniques and/or automated peptide synthesizers, or by recombinant means. In certain instances, the αᵥβ₆-BP may be synthesized using solid phase strategies on an automated multiple peptide synthesizer (Abimed AMS 422) using 9-fluorenylmethyloxycarbonyl (Fmoc) chemistry. The αᵥβ₆-BP can then be purified by reversed phase-HPLC and lyophilized. The αᵥβ₆-BP may alternatively be prepared by cleavage of a longer peptide or full-length protein sequence. For example, a fragment containing the αᵥβ₆ integrin-binding domain of fibronectin, tenascin, vitronectin, the latency associated peptide (LAP) of TGF-β, or viral capsid protein (VP1) of foot-and-mouth disease virus (FMDV) can be isolated by cleavage of the full-length protein.

In other embodiments, the αᵥβ₆-BP component of the conjugates may be cyclized. Methods are well known in the art for introducing cyclic structures into peptides to select and provide conformational constraints to the structure that result in enhanced stability. For example, a C- or N-terminal cysteine can be added to the αᵥβ₆-BP, so that when oxidized the peptide will contain a disulfide bond, generating a cyclic peptide. Other peptide cyclization methods include the formation of thioethers and carboxyl- and amino-terminal amides and esters. A number of synthetic techniques have been developed to generate synthetic circular peptides (see, e.g., Tam et al., Protein Sci., 7:1583-1592 (1998); Romanovskis et al., J. Pept. Res., 52: 356-374 (1998); Camarero et al., J. Amer. Chem. Soc., 121: 5597-5598 (1999); Valero et al., J. Pept. Res., 53(1): 56-67 (1999)). Generally, the role of cyclizing peptides is two-fold: (1) to reduce hydrolysis in vivo; and (2) to thermodynamically destabilize the unfolded state and promote secondary structure formation.

### Imaging and Detection Methods for Virus-Related Lung Damage

In some embodiments, the conjugates described herein are utilized in methods of imaging lung tissue. Such imaging includes detection by Magnetic Resonance Imaging (MM), Magnetic Resonance Spectroscopy (MRS), Single Photon Emission Computerized Tomography (SPECT), Positron Emission Tomography (PET), or optical imaging.

In some embodiments, the method relates to the in vivo imaging of a lung tissue or a portion thereof, the method comprising administering to a subject in need of such imaging, an αᵥβ₆-BP conjugate described herein or a composition thereof and (b) detecting the conjugate to determine where the conjugate is concentrated in the lung tissue of the subject. In some embodiments, the subject suffers from a respiratory viral infection, such as a SARS coronavirus, including SARS-CoV or SARS-CoV-2. In some embodiments, the subject is a COVID-19 patient. In some embodiments, the subject is suspected to have been infected by a SARS coronavirus. In some embodiments, the subject has been exposed to or is suspected of having been exposed to a SARS coronavirus.

The methods described herein include methods of imaging virus-related lung damage, such as lung damage caused by a SARS coronavirus infection, including infection by SARS-CoV or SARS-CoV-2. The method includes (a) administering to a subject an αᵥβ₆-BP conjugate described herein where the subject has been exposed to or is suspected of exposure to a respiratory virus causative of lung damage; and (b) detecting the conjugate in lung tissue (e.g., in the subject) to determine where the location and/or concentration of the conjugate in lung tissue and thereby imaging the lung damage. In some embodiments, the subject is infected or has been exposed to or is suspected of having been exposed to a SARS coronavirus, such as SARS-CoV or SARS-CoV-2. In some aspects, the subject has been diagnosed as a COVID-19 patient, based on symptoms exhibited and/or a diagnostic test for SARS-CoV-2. In some aspects, the subject is being quarantined or has been quarantined for exposure or expected exposure to SARS-CoV-2. In some embodiments, the subject is infected with SARS-CoV-2 and exhibits one or more symptoms of a COVID-19 disease. In some aspects the subject suffers from mild, moderate, or severe COVID-19 disease. In some embodiments, the subject is symptomatic for COVID-19. In some embodiments, the subject is asymptomatic for COVID-19. In some embodiments, the subject is a COVID long-hauler. In some embodiments, the subject exhibits one or more symptoms of lung damage such as shortness of breath or a need for continued oxygen treatment. In some embodiments, the subject exhibits one or more symptoms such as coughing, ongoing fatigue, body aches, joint pain, loss of taste and smell, difficulty sleeping, headaches or brain fog.

In some embodiments, imaging is performed at one or more time points after a subject is designated as infected with SARS-CoV-2 and/or one or more time points after a subject is suspected of having been infected with SARS-CoV-2. In some embodiments, imaging with an αᵥβ₆-BP conjugate is performed 1 day, 2 days, 3 days, 4 days, 5 days, 6 days, 7 days, 8 days, 9 days, 10 days, 11, days, 12 days, 13 days, 14 days, 15 days, 16 days, 17 days, 18 days, 19 days, 20 days, 21 days or more than 21 days after infection or suspected infection with SARS-CoV-2.

In some embodiments, imaging is performed at one or more time points as a subject is recovering from or has recovered from COVID-19. In some aspects, the imaging is performed 1 day, 2 days, 3 days, 4 days, 5 days, 6 days, 7 days, 8 days, 9 days, 10 days, 11, days, 12 days, 13 days, 14 days, 15 days, 16 days, 17 days, 18 days, 19 days, 20 days, 21 days or more than 21 days after the subject has recovered from COVID-19 and/or performed 1 day, 2 days, 3 days, 4 days, 5 days, 6 days, 7 days, 8 days, 9 days, 10 days, 11, days, 12 days, 13 days, 14 days, 15 days, 16 days, 17 days, 18 days, 19 days, 20 days, 21 days or more than 21 days after the subject has begun recovery from COVID-19.

In some embodiments, the administration of the conjugate and subsequent imaging step are repeated during the course of infection with the respiratory virus (e.g., SARS coronavirus) at two or more time points. In some embodiments, the administration of the conjugate and subsequent imaging step are repeated during the course of recovery at two or more time points. In some embodiments, the steps are repeated at least 2, 3, 4, 5, 6, 7, 8, 9, 10 or more than 10 times over the course of infection, the course of recovery, or the course of infection and recovery.

In some embodiments, the imaging methods described herein utilize an αᵥβ₆-BP conjugate described herein to image viral-induced or virally-caused lung damage. Such damage includes pulmonary fibrosis.

CT scans of lung tissue from COVID-19 patients have revealed ground-glass-like opacifications. For example, histopathology may reveal diffuse alveolar damage, denuded alveolar lining cells with reactive type II pneumocyte hyperplasia, intra-alveolar fibrinous exudates, loose interstitial fibrosis and chronic inflammatory infiltrates, intra-alveolar loose fibrous plugs of organizing pneumonia, and intra-alveolar organizing fibrin in foci.

The methods described herein utilizing an αᵥβ₆-BP conjugate for imaging may provide a more sensitive and/or more accurate image of such lung damage. For example, improved sensitivity may include the ability to detect infection or symptoms of infection earlier in time than CT or other available methods. In some embodiments, increased sensitivity may include the ability to localize the damage within lung tissue to specific regions of the lung. In some embodiments, the sensitivity of the methods can include quantitation of the amount of lung damage in an area of lung tissue or change of lung damage over time (such as increased damage if infection progresses and/or does not diminish, or decreased damage as a subject recovers from COVID-19).

In some embodiments, the methods of imaging with an αᵥβ₆-BP conjugate described herein are used to monitor a “long-hauler” syndrome in a subject. Long-hauler refers to COVID-19 patients who experience lingering symptoms. Those individuals are often referred to as “COVID long-haulers” or “long-haulers” and the condition is referred to as COVID-19 syndrome or “long COVID.” For COVID long-haulers, persistent symptoms often include brain fog, fatigue, headaches, dizziness and shortness of breath, among others. Such patients may require ongoing oxygen treatment, even after hospital discharge, and such oxygen treatments may continue for weeks to months because of permanent damage to the lungs. Such patients may also experience cardiac symptoms, persistent kidney dysfunction as well as newly diagnosed diabetes or worsened control of diabetes. In many cases, long-haulers test negative for the SARS-CoV-2 virus even when symptoms persist. In some embodiments, a long-hauler is imaged with an αᵥβ₆-BP conjugate over a period of time, such as over 2 weeks, 4 weeks, 6 weeks, 8 weeks, 2 months, 3 months, 4 months, 5 months, 6 months, 7 months, 8 months, 9 months, 10 months, 11 months, 12 months, 1 year, 2 years, 3 years, or more post infection. In some embodiments, blood biomarkers such cytokines, interleukins, and integrins are monitored in an overlapping or similar periodicity and compared with the images obtained with the αᵥβ₆-BP conjugate. In particular embodiments, the imaging is compared to standard of care imaging such as CT. In particular embodiments, lung functionality is tested and compared or correlated with the images obtained with the αᵥβ₆-BP conjugate.

The imaging methods described herein include administering an αᵥβ₆-BP conjugate to a subject followed by a step of detecting the conjugate in the subject's lung or a portion thereof. In some embodiments, the conjugate is administered to the subject by intravenous injection. In some aspects, the imaging (detection) step is performed between 10 minutes and 180 minutes after administering the conjugate. In some aspects, the imaging (detection) step is performed at about 15 minutes, 30 minutes, 60 minutes, 75 minutes, 90 minutes or 120 minutes after administering the conjugate. In some embodiments, a subject undergoes one or rounds of the administration plus imaging over time, for example, to determine the extent of the regression or progression of lung damage over time.

The conjugates described herein can be administered either systemically or locally prior to the imaging procedure. Generally, the conjugates are administered in doses effective to achieve the desired optical image of a tumor, tissue, or organ. Such doses may vary widely, depending upon the particular conjugate employed, the tumor, tissue, or organ subjected to the imaging procedure, the imaging equipment being used, and the like.

A detectable response generally refers to a change in, or occurrence of, an optical signal that is detectable either by observation or instrumentally. In certain instances, the detectable response is radioactivity (i.e., radiation), including alpha particles, beta particles, nucleons, electrons, positrons, neutrinos, and gamma rays emitted by a radioactive substance such as a radionuclide. In certain other instances, the detectable response is fluorescence or a change in fluorescence, e.g., a change in fluorescence intensity, fluorescence excitation or emission wavelength distribution, fluorescence lifetime, and/or fluorescence polarization. In some aspects, the degree and/or location of labeling in a subject or sample can be compared to a standard or control (e.g., healthy tissue or organ).

When used in the imaging methods described herein, the conjugates described herein typically have an imaging agent covalently or non-covalently attached to one or more of the αᵥβ₆-BP or the first or second PEG moiety. Suitable imaging agents include, but are not limited to, radionuclides, detectable tags, fluorophores, fluorescent proteins, enzymatic proteins, and the like. In some embodiments, the imaging agent can be directly attached to the αᵥβ₆-BP or PEG portion of the conjugate via covalent attachment of the imaging agent to a primary amine group present in the peptide or PEG moiety. An imaging agent can also be bound to the αᵥβ₆-BP or PEG portion of the conjugate via non-covalent interactions (e.g., ionic bonds, hydrophobic interactions, hydrogen bonds, Van der Waals forces, dipole-dipole bonds, etc.).

In some embodiments, the conjugate is radiolabeled with a radionuclide by directly attaching the radionuclide to one or more of the αᵥβ₆-BP or the first or second PEG moiety of the conjugate. In certain other instances, a benzoyl group labeled with the radionuclide is directly attached to the αᵥβ₆-BP or PEG portion of the conjugate. For example, 4-[¹⁸F]-fluorobenzoic acid (“[¹⁸F]FBA”) or 4-[¹⁹F]-fluorobenzoic acid (“[¹⁹F]FBA”) can be used to radiolabel the conjugates. In further instances, the radionuclide is bound to a chelating agent or chelating agent-linker attached to the conjugate. Suitable radionuclides for direct conjugation include, without limitation, ¹⁸F, ¹⁹F, ¹²⁴I, ¹²⁵I, ¹³¹I, and mixtures thereof. Suitable radionuclides for use with a chelating agent include, without limitation, ⁴⁷Sc, 64Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, but are not limited to, DOTA, NOTA, NOTA-TCO, BAD, TETA, DTPA, EDTA, NTA, HDTA, their phosphonate analogs, and mixtures thereof. One of skill in the art will be familiar with methods for attaching radionuclides, chelating agents, and chelating agent-linkers to the conjugates. In particular, attachment can be conveniently accomplished using, for example, commercially available bifunctional linking groups (generally heterobifunctional linking groups) that can be attached to a functional group present in a non-interfering position on the conjugate and then further linked to a radionuclide, chelating agent, or chelating agent-linker.

Non-limiting examples of fluorophores or fluorescent dyes suitable for use as imaging agents include Alexa Fluor® dyes (Invitrogen Corp.; Carlsbad, Calif.), fluorescein, fluorescein isothiocyanate (FITC), Oregon Green™; rhodamine, Texas red, tetrarhodamine isothiocynate (TRITC), CyDye™ fluors (e.g., Cy2, Cy3, Cy5), and the like.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al., Mol. Microbiol., 55:1767-1781 (2005), the GFP variant described in Crameri et al., Nat. Biotechnol., 14:315-319 (1996), the cerulean fluorescent proteins described in Rizzo et al., Nat. Biotechnol., 22:445 (2004) and Tsien, Annu. Rev. Biochem., 67:509 (1998), and the yellow fluorescent protein described in Nagal et al., Nat. Biotechnol., 20:87-90 (2002). DsRed variants are described in, e.g., Shaner et al., Nat. Biotechnol., 22:1567-1572 (2004), and include mStrawberry, mCherry, mOrange, mBanana, mHoneydew, and mTangerine. Additional DsRed variants are described in, e.g., Wang et al., Proc. Natl. Acad. Sci. U.S.A., 101:16745-16749 (2004) and include mRaspberry and mPlum. Further examples of DsRed variants include mRFPmars described in Fischer et al., FEBS Lett., 577:227-232 (2004) and mRFPruby described in Fischer et al., FEBS Lett., 580:2495-2502 (2006).

In other embodiments, the imaging agent that is bound to a conjugate comprises a detectable tag such as, for example, biotin, avidin, streptavidin, or neutravidin. In further embodiments, the imaging agent comprises an enzymatic protein including, but not limited to, luciferase, chloramphenicol acetyltransferase, β-galactosidase, β-glucuronidase, horseradish peroxidase, xylanase, alkaline phosphatase, and the like.

For methods utilizing conjugates where the imaging agent includes a radionuclide, any device or method known or available for detecting the radioactive emissions of radionuclides in a subject is suitable for use with the conjugates described herein. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled conjugate. Positron emission tomography (PET) is another suitable technique for detecting radiation in a subject. Furthermore, U.S. Pat. No. 5,429,133 describes a laparoscopic probe for detecting radiation concentrated in solid tissue tumors. Miniature and flexible radiation detectors intended for medical use are produced by Intra-Medical LLC (Santa Monica, Calif.). Magnetic Resonance Imaging (MRI) or any other imaging technique known to one of skill in the art is also suitable for detecting the radioactive emissions of radionuclides. Regardless of the method or device used, such detection is aimed at determining where the conjugate is concentrated in a subject, with such concentration being an indicator of the location of a tumor or tumor cells.

Non-invasive fluorescence imaging of animals and humans can also provide in vivo diagnostic or prognostic information and be used in a wide variety of clinical specialties. For instance, techniques have been developed over the years for simple ocular observations following UV excitation to sophisticated spectroscopic imaging using advanced equipment (see, e.g., Andersson-Engels et al., Phys. Med. Biol., 42:815-824 (1997)). Specific devices or methods known in the art for the in vivo detection of fluorescence, e.g., from fluorophores or fluorescent proteins, include, but are not limited to, in vivo near-infrared fluorescence (see, e.g., Frangioni, Curr. Opin. Chem. Biol., 7:626-634 (2003)), the Maestro™ in vivo fluorescence imaging system (Cambridge Research & Instrumentation, Inc.; Wobum, Mass.), in vivo fluorescence imaging using a flying-spot scanner (see, e.g., Ramanujam et al., IEEE Transactions on Biomedical Engineering, 48:1034-1041 (2001)), and the like.

Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, and signal amplification using photomultiplier tubes.

### Therapeutic Methods for Treating Virus-Related Lung Damage

In some aspects, the conjugates described herein are utilized in methods of treating viral-damaged lung tissue. The conjugates for such treatment may comprise an αᵥβ₆-BP covalently linked to a therapeutic agent, such as a small molecule. In some embodiments, the conjugates for such treatment comprise an αᵥβ₆-BP covalently linked to an anti-fibrotic agent, an anti-inflammatory agent, or an anti-viral agent. In particular embodiments, the conjugate comprises nintedanib, pirfenidone, remdesivir or hydroxychloroquine.

In some embodiments, the method comprises administering to a subject in need of treatment, such as a subject suffering from or diagnosed with lung damage from a respiratory viral infection, an αᵥβ₆-BP therapeutic conjugate described herein or a pharmaceutical composition thereof. In some embodiments, the subject suffers from a respiratory viral infection, such as a SARS coronavirus, including SARS-CoV or SARS-CoV-2. In some embodiments, the subject is a COVID-19 patient. In some embodiments, the subject is suspected to have been infected by a SARS coronavirus. In some embodiments, the subject has been exposed to or is suspected of having been exposed to a SARS coronavirus.

The methods described herein include methods of treating virus-related lung damage, such as lung damage caused by a SARS coronavirus infection, including infection by SARS-CoV or SARS-CoV-2. The method includes administering to a subject a therapeutically effective amount of an αᵥβ₆-BP therapeutic conjugate described herein where the subject has been exposed to or is suspected of exposure to a respiratory virus causative of lung damage. In some embodiments, the subject is infected or has been exposed to or is suspected of having been exposed to a SARS coronavirus, such as SARS-CoV or SARS-CoV-2. In some embodiments, the subject has been diagnosed as a COVID-19 patient, based on symptoms exhibited and/or a diagnostic test for SARS-CoV-2. In some embodiments, the subject is infected with SARS-CoV-2 and exhibits one or more symptoms of a COVID-19 disease. In some embodiments, the subject suffers from mild, moderate, or severe COVID-19 disease. In some embodiments, the subject is symptomatic for COVID-19. In some embodiments, the subject is asymptomatic for COVID-19. In some embodiments, the subject is a COVID long-hauler. In some embodiments, the subject exhibits one or more symptoms of lung damage such as shortness of breath or a need for continued oxygen treatment. In some embodiments, the subject exhibits one or more symptoms such as coughing, ongoing fatigue, body aches, joint pain, loss of taste and smell, difficulty sleeping, headaches or brain fog.

In particular embodiments, the subject has been diagnosed with lung damage by imaging the lungs prior to treatment. In some embodiments, the subject is imaged with an αᵥβ₆-BP imaging conjugate described herein prior to treatment with an αᵥβ₆-BP therapeutic conjugate described herein.

In some embodiments, the αᵥβ₆-BP therapeutic conjugate is administered within 1, 2, 3, 4, 5, 6, or 7 days or within 1, 2, 3, 4, 5, 6, or 7 weeks of initial infection. In some embodiments, the αᵥβ₆-BP therapeutic conjugate is administered within 1, 2, 3, 4, 5, 6, or 7 days or within 1, 2, 3, 4, 5, 6, or 7 weeks of the onset of symptoms. In some embodiments, the αᵥβ₆-BP therapeutic conjugate is administered after lung damage is diagnosed by a lung image or other diagnostic criteria. In some aspects, the αᵥβ₆-BP therapeutic conjugate is administered once, twice, or multiple times over a course of treatment.

### Pharmaceutical Compositions

In some embodiments of the compositions and methods described herein, the αᵥβ₆-BP conjugate is administered in a pharmaceutical composition. Such compositions typically include a conventional pharmaceutical carrier or excipient and may additionally include other medicinal agents, carriers, adjuvants, diluents, tissue permeation enhancers, solubilizers, and the like. In particular embodiments, the composition contains about 0.01% to about 90%, about 0.1% to about 75%, about 0.1% to 50%, or about 0.1% to 10% by weight of an αᵥβ₆-BP conjugate or a combination thereof, with the remainder consisting of suitable pharmaceutical carrier and/or excipients. Appropriate excipients can be tailored to the particular composition and route of administration by methods well known in the art. See, e.g., REMINGTON'S PHARMACEUTICAL SCIENCES, supra.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The compositions can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; coloring agents; and flavoring agents. The compositions may also comprise biodegradable polymer beads, dextran, and cyclodextrin inclusion complexes.

Administration of the αᵥβ₆-BP conjugates described herein with a suitable pharmaceutical excipient as necessary can be carried out via any of the accepted modes of administration. Thus, administration can be, for example, intravenous, topical, subcutaneous, transcutaneous, transdermal, intramuscular, oral, intra-joint, parenteral, intra-arteriole, intradermal, intraventricular, intracranial, intraperitoneal, intralesional, intranasal, rectal, vaginal, or by inhalation. In some embodiments here, the conjugate is administered intravenously.

For oral administration, the compositions can be in the form of tablets, lozenges, capsules, emulsions, suspensions, solutions, syrups, sprays, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the pharmaceutical compositions take the form of a pill, tablet, or capsule, and thus, the composition can contain, along with the conjugate or combination of conjugates, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. The conjugates can also be formulated into a suppository disposed, for example, in a polyethylene glycol (PEG) carrier.

Liquid compositions can be prepared by dissolving or dispersing a conjugate or a combination of conjugates and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. The conjugates can also be formulated into a retention enema.

For topical administration, the compositions herein can be in the form of emulsions, lotions, gels, creams, jellies, solutions, suspensions, ointments, and transdermal patches. For delivery by inhalation, the composition can be delivered as a dry powder or in liquid form via a nebulizer. For parenteral administration, the compositions can be in the form of sterile injectable solutions and sterile packaged powders. Preferably, injectable solutions are formulated at a pH of about 4.5 to about 7.5.

The compositions described herein can also be provided in a lyophilized form. Such compositions may include a buffer, e.g., bicarbonate, for reconstitution prior to administration, or the buffer may be included in the lyophilized composition for reconstitution with, e.g., water. The lyophilized composition may further comprise a suitable vasoconstrictor, e.g., epinephrine. The lyophilized composition can be provided in a syringe, optionally packaged in combination with the buffer for reconstitution, such that the reconstituted composition can be immediately administered to a patient.

### Kits for Imaging and Detection

Also provided herein are kits to facilitate and/or standardize the use of the conjugates and compositions described herein, as well as to facilitate the methods described herein. Materials and reagents to carry out these various methods can be provided in kits to facilitate execution of the methods. As used herein, the term “kit” includes a combination of articles that facilitates a process, assay, analysis, or manipulation. In particular, kits comprising the conjugates or compositions of the conjugates can be stored and/or shipped to locations where the imaging is to be performed such as to a clinic or hospital.

Kits can contain chemical reagents as well as other components. In addition, the kits containing the conjugates herein can include, without limitation, instructions to the kit user (e.g., directions for use of the conjugate or composition for use in imaging subjects infected by or suspected of infection by a SARS coronavirus), Kits of the conjugates or compositions thereof can also be packaged for convenient storage and safe shipping, for example, as ampules or other vials packaged in a box having a lid.

### Definitions

The terms “integrin-binding peptide” and “peptide that binds to an integrin” refer to the binding/interaction of a peptide motif in the conjugate which shows the capacity of specific interaction with a specific integrin or a specific group of integrins. The term “αᵥβ₆-binding peptide” refers to the binding/interaction of a peptide motif in the conjugate which shows the capacity of specific interaction with αᵥβ₆ integrin.

In some embodiments, the terms refer to the ability of a peptide or a portion thereof to interact with and/or bind to a target integrin and without cross-reacting with molecules of similar sequences or structures. In some instances, a peptide specifically binds to a target integrin when it binds to the target integrin with a substantially lower dissociation constant (i.e., tighter binding) than a molecule of similar sequence or structure. For example, in certain instances, a specific binding occurs when the peptide binds to the target integrin with an about 2, 3, 4, 5, 6, 8, 10, 15, 20, 25, 30, 40, 50, 100, or 1000-fold or greater affinity than a related molecule. The binding of the peptide to a site on the target integrin may occur via intermolecular forces such as ionic bonds, hydrogen bonds, hydrophobic interactions, dipole-dipole bonds, and/or Van der Waals forces. Cross-reactivity may be tested, for example, by assessing binding of the peptide under conventional conditions to the target integrin as well as to a number of more or less (e.g., structurally and/or functionally) closely related molecules. These methods may include, without limitation, binding studies, blocking and competition studies with closely related molecules, FACS analysis, surface plasmon resonance (e.g., with BIAcore), analytical ultracentrifugation, isothermal titration calorimetry, fluorescence anisotropy, fluorescence spectroscopy, radiolabeled ligand binding assays, and combinations thereof.

As used herein, the term “PEGylation” refers to the process of covalently coupling a polyethylene glycol (PEG) molecule to another molecule, e.g., a peptide, polypeptide, protein, antibody, and the like, which is then referred to as “PEGylated.” As a non-limiting example, an integrin-binding peptide may be PEGylated at both the amino-terminus and the carboxyl terminus with monodisperse PEG molecules having a defined chain length to generate bi-terminal PEGylated peptide conjugates. Monodisperse PEG molecules typically comprise discrete molecular weights with an exactly defined number of repeating ethylene glycol units. PEG moieties suitable for use are commercially available from Polypure AS (Oslo, Norway), which supplies monodisperse PEG molecules and PEG derivatives thereof consisting of substantially one oligomer only (e.g., greater than about 90%, 91%, 92%, 93%, 94%, 95%, 96%, 97%, 98%, or 99% oligomer purity). In particular embodiments, the integrin-binding peptide is PEGylated at both ends with a single type or mixtures of different types of monodisperse PEG moieties having a molecular weight of less than about 5,000 daltons (Da) (e.g., less than about 5,000, 4,000, or 3,000 Da), such as, e.g., PEG₁₁, PEG₁₂ (PEG 800), PEG₂₈ (PEG 1500), and/or (PEG₂₈)₂ (PEG 1500×2).

A “peptidomimetic” refers to a chemical compound having a structure that is different from the general structure of an existing peptide, but that functions in a manner similar to the existing peptide, e.g., by mimicking the biological activity of that peptide. Peptidomimetics typically comprise naturally-occurring amino acids and/or unnatural amino acids, but can also comprise modifications to the peptide backbone. Peptidomimetics can exhibit increased affinity, specificity, and/or stability compared to an existing peptide.

The term “amino acid” includes naturally-occurring α-amino acids and their stereoisomers, as well as unnatural amino acids and their stereoisomers. “Stereoisomers” of amino acids refers to mirror image isomers of the amino acids, such as L-amino acids or D-amino acids. For example, a stereoisomer of a naturally-occurring amino acid refers to the mirror image isomer of the naturally-occurring amino acid, i.e., the D-amino acid.

Naturally-occurring amino acids are those encoded by the genetic code, as well as those amino acids that are later modified, e.g., α-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” are unnatural amino acids that have the same basic chemical structure as naturally-occurring amino acids, i.e., an a carbon that is bound to a hydrogen, a carboxyl group, an amino group, but have modified R (i.e., side-chain) groups.

Non-limiting examples of unnatural amino acids include 1-aminocyclopentane-1-carboxylic acid (Acp), 1-aminocyclobutane-1-carboxylic acid (Acb), 1-aminocyclopropane-1-carboxylic acid (Acpc), citrulline (Cit), homocitrulline (HoCit), α-aminohexanedioic acid (Aad), 3-(4-pyridyl)alanine (4-Pal), 3-(3-pyridyl)alanine (3-Pal), propargylglycine (Pra), α-aminoisobutyric acid (Aib), α-aminobutyric acid (Abu), norvaline (Nva), α,β-diaminopropionic acid (Dpr), α,γ-diaminobutyric acid (Dbu), α-tert-butylglycine (Bug), 3,5-dinitrotyrosine (Tyr(3,5-di NO₂)), norleucine (Nle), 3-(2-naphthyl)alanine (Nal-2), 3-(1-naphthyl)alanine (Nal-1), cyclohexylalanine (Cha), di-n-propylglycine (Dpg), cyclopropylalanine (Cpa), homoleucine (Hle), homoserine (HoSer), homoarginine (Har), homocysteine (Hcy), methionine sulfoxide (Met(O)), methionine methylsulfonium (Met (S-Me)), α-cyclohexylglycine (Chg), 3-benzo-thienylalanine (Bta), taurine (Tau), hydroxyproline (Hyp), O-benzyl-hydroxyproline (Hyp(Bzl)), homoproline (HoPro), β-homoproline (βHoPro), thiazolidine-4-carboxylic acid (Thz), nipecotic acid (Nip), isonipecotic acid (IsoNip), 3-carboxymethyl-1-phenyl-1,3,8-triazaspiro[4,5]decan-4-one (Cptd), tetrahydro-isoquinoline-3-carboxylic acid (3-Tic), 5H-thiazolo[3,2-a]pyridine-3-carboxylic acid (Btd), 3-aminobenzoic acid (3-Abz), 3-(2-thienyl)alanine (2-Thi), 3-(3-thienyl)alanine (3-Thi), α-aminooctanedioc acid (Asu), diethylglycine (Deg), 4-amino-4-carboxy-1,1-dioxo-tetrahydrothiopyran (Acdt), 1-amino-1-(4-hydroxycyclohexyl) carboxylic acid (Ahch), 1-amino-1-(4-ketocyclohexyl)carboxylic acid (Akch), 4-amino-4-carboxytetrahydropyran (Actp), 3-nitrotyrosine (Tyr(3-NO2)), 1-amino-1-cyclohexane carboxylic acid (Ach), 1-amino-1-(3-piperidinyl)carboxylic acid (3-Apc), 1-amino-1-(4-piperidinyl)carboxylic acid (4-Apc), 2-amino-3-(4-piperidinyl) propionic acid (4-App), 2-aminoindane-2-carboxylic acid (Aic), 2-amino-2-naphthylacetic acid (Ana), (2S,5R)-5-phenylpyrrolidine-2-carboxylic acid (Ppca), 4-thiazoylalanine (Tha), 2-aminooctanoic acid (Aoa), 2-aminoheptanoic acid (Aha), ornithine (Om), azetidine-2-carboxylic acid (Aca), α-amino-3-chloro-4,5-dihydro-5-isoazoleacetic acid (Acdi), thiazolidine-2-carboxylic acid (Thz(2-COOH)), allylglycine (Agl), 4-cyano-2-aminobutyric acid (Cab), 2-pyridylalanine (2-Pal), 2-quinoylalanine (2-Qal), cyclobutylalanine (Cba), a phenylalanine analog, derivatives of lysine, ornithine (Om) and α,γ-diaminobutyric acid (Dbu), stereoisomers thereof, and combinations thereof (see, e.g., Liu et al., Anal. Biochem., 295:9-16 (2001)). As such, the unnatural α-amino acids are present either as unnatural L-α-amino acids, unnatural D-α-amino acids, or combinations thereof.

“Amino acid mimetics” are chemical compounds that have a structure that is different from the general chemical structure of an amino acid, but that function in a manner similar to a naturally-occurring amino acid. Suitable amino acid mimetics include, without limitation, β-amino acids and γ-amino acids. In R-amino acids, the amino group is bonded to the β-carbon atom of the carboxyl group such that there are two carbon atoms between the amino and carboxyl groups. In α-amino acids, the amino group is bonded to the γ-carbon atom of the carboxyl group such that there are three carbon atoms between the amino and carboxyl groups. Suitable R groups for β- or γ-amino acids include, but are not limited to, side-chains present in naturally-occurring amino acids and unnatural amino acids.

“N-substituted glycines” are unnatural amino acids based on glycine, where an amino acid side-chain is attached to the glycine nitrogen atom. Suitable amino acid side-chains (e.g., R groups) include, but are not limited to, side chains present in naturally-occurring amino acids and side-chains present in unnatural amino acids such as amino acid analogs. Non-limiting examples of N-substituted glycines include N-(2-aminoethyl)glycine, N-(3-aminopropyl)glycine, N-(2-methoxyethyl)glycine, N-benzylglycine, (S)—N-(1-phenylethyl)glycine, N-cyclohexylmethylglycine, N-(2-phenylethyl)glycine, N-(3-phenylpropyl)glycine, N-(6-aminogalactosyl)glycine, N-(2-(3′-indolylethyl)glycine, N-(2-(p-methoxyphenylethyl))glycine, N-(2-(p-chlorophenylethyl)glycine, and N-[2-(p-hydroxyphenylethyl)]glycine. N-substituted glycine oligomers, referred to herein as “peptoids,” have been shown to be protease resistant (see, e.g., Miller et al., Drug Dev. Res., 35:20-32 (1995)).

Amino acids may be referred to herein by either their commonly known three letter symbols or by the one-letter symbols recommended by the IUPAC-IUB Biochemical Nomenclature Commission. For example, an L-amino acid may be represented herein by its commonly known three letter symbol (e.g., Arg for L-arginine) or by an upper-case one-letter amino acid symbol (e.g., R for L-arginine). A D-amino acid may be represented herein by its commonly known three letter symbol (e.g., D-Arg for D-arginine) or by a lower-case one-letter amino acid symbol (e.g., r for D-arginine).

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another (see, e.g., Creighton, Proteins, 1993):

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M).

The term “peptide” refers to a compound made up of a single chain of D- or L-amino acids or a mixture of D- and L-amino acids joined by peptide bonds. Generally, peptides are about 2 to about 50 amino acids in length. As non-limiting examples, the peptides present in the conjugates described herein are between about 5 to about 45 amino acids in length, between about 8 to about 45 amino acids in length, between about 8 to about 25 amino acids in length, between about 8 to about 20 amino acids in length, between about 12 to about 45 amino acids in length, between about 12 to about 30 amino acids in length, about 8 amino acids in length, or about 20 amino acids in length.

A “cyclic peptide” refers to a peptide in which the amino-terminus of the peptide or a side-chain on the peptide having a free amino group (e.g., lysine) is joined by a peptide bond to the carboxyl-terminus of the peptide or a side-chain on the peptide having a free carboxyl group (e.g., aspartic acid, glutamic acid). However, one skilled in the art will appreciate that heterodetic cyclic peptides formed by disulfide, ester, or ether bonds are also useful for the conjugates herein.

The term “helix-promoting residue” includes amino acids with a conformational preference greater than 1.0 for being found in the middle of an α-helix (see, e.g., Creighton, Proteins, 1993; and Pace et al., Biophysical 1, 75:422-427 (1998)). However, non-orthodox helix-promoting combinations of amino acids may also be utilized if they enhance the specificity and/or affinity of binding to a target integrin, e.g., αᵥβ₆ integrin.

As used herein, the term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a conjugate or composition is administered at the same time, just prior to, or just after the administration of a second agent, such as a therapeutic agent, an additional imaging agent or other compound.

The term “radionuclide” is intended to include any nuclide that exhibits radioactivity. A “nuclide” refers to a type of atom specified by its atomic number, atomic mass, and energy state, such as carbon 14 (¹⁴C). “Radioactivity” refers to the radiation, including alpha particles, beta particles, nucleons, electrons, positrons, neutrinos, and gamma rays, emitted by a radioactive substance. Examples of radionuclides suitable for use in the conjugates described herein include, but are not limited to, fluorine 18 (¹⁸F), fluorine 19 (¹⁹F), phosphorus 32 (³²P), scandium 47 (⁴⁷Sc), cobalt 55 (⁵⁵Co), copper 60 (⁶⁰Cu), copper 61 (⁶¹Cu), copper 62 (⁶²Cu), copper 64 (⁶⁴Cu), gallium 66 (⁶⁶Ga), copper 67 (⁶⁷Cu), gallium 67 (⁶⁷Ga), gallium 68 (⁶⁸Ga), rubidium 82 (⁸²Rb), yttrium 86 (⁸⁶Y), yttrium 87 (⁸⁷Y), strontium 89 (⁸⁹Sr), yttrium 90 (⁹⁰Y) rhodium 105 (¹⁰⁵Rh) silver 111 (¹¹¹Ag), indium 111 (¹¹¹In), iodine 124 (¹²⁴I), iodine 125 (¹²⁵I), iodine 131 (¹³¹I), tin 117m (^{117m}Sn), technetium 99m (^{99m}Tc), promethium 149 (¹⁴⁹Pm), samarium 153 (¹⁵³Sm), holmium 166 (¹⁶⁶Ho), lutetium 177 (¹⁷⁷Lu), rhenium 186 (¹⁸⁶Re), rhenium 188 (¹⁸⁸Re), thallium 201 (²⁰¹Tl), astatine 211 (²¹¹At), and bismuth 212 (²¹²Bi). As used herein, the “m” in ^{117m} and ^{99m}Tc stands for the meta state. Additionally, naturally-occurring radioactive elements such as uranium, radium, and thorium, which typically represent mixtures of radioisotopes, are suitable examples of radionuclides. ⁶⁷Cu, ¹³¹I, ¹⁷⁷Lu, and ¹⁸⁶Re are beta- and gamma-emitting radionuclides. ²¹²Bi is an alpha- and beta-emitting radionuclide. ²¹¹At is an alpha-emitting radionuclide. ³²P, ⁴⁷Sc, ⁸⁹Sr, ⁹⁰Y, ¹⁰⁵Rh, ¹¹¹Ag, ^{17m}Sm, ¹⁴⁹ Pm, ¹⁵³Sm, ¹⁶⁶Ho, and ¹⁸⁸Re are examples of beta-emitting radionuclides. ⁶⁷Ga, ¹¹¹In, ^{99m}Tc, and ²⁰¹Tl are examples of gamma-emitting radionuclides. ⁵⁵Co, ⁶⁰Cu, ⁶¹Cu, ⁶²Cu, ⁶⁶Ga, ⁶⁸Ga, ⁸²Rb, and ⁸⁶Y are examples of positron-emitting radionuclides. ⁶⁴Cu is a beta- and positron-emitting radionuclide.

The term “subject” or “patient” typically refers to humans, but can also include other animals such as, e.g., other primates, rodents, canines, felines, equines, ovines, porcines, and the like.

The term “unit dosage form” refers to physically discrete units suitable as unitary dosages for human subjects and other mammals (e.g., dogs), each unit containing a predetermined quantity of active material calculated to produce the desired effects, alone or as formulated with a suitable pharmaceutical excipient (e.g., an ampoule). In addition, more concentrated compositions may be prepared, from which the more dilute unit dosage compositions may then be produced. The more concentrated compositions thus will contain substantially more than, e.g., at least 1, 2, 3, 4, 5, 6, 7, 8, 9, 10, or more times the amount of a conjugate or a combination of conjugates. Methods for preparing such dosage forms are known to those skilled in the art (see, for example, REMINGTON'S PHARMACEUTICAL SCIENCES, 18TH ED., Mack Publishing Co., Easton, Pa. (1990)).

### EXAMPLES

The following examples are included for illustrative purposes only and are not intended to limit the scope of the invention.

### Example 1: Imaging of Lung Fibrosis in Non-Viral Conditions

The ¹⁸F-αᵥβ₆-BP was used in a first-in-human study demonstrating the ability to image both primary and metastatic disease (NCT03164486). 26 subjects including patients with a prior diagnosis of breast, colon, lung or pancreas cancer were enrolled. PET images showed low background uptake in normal brain, lungs, liver and osseous skeleton which are common sites of metastatic disease. Furthermore, sub-centimeter metastasis to these organs were detected using ⁸F-αᵥβ₆-BP.

All participants were scanned at 30, 60, 120, and 180 minutes post ¹⁸F-αᵥβ₆-BP injection. Reconstruction of the images was performed using OSEM. PET/CT image review was performed in multiple formats including axial, sagittal, coronal, fused, unfused, corrected and uncorrected datasets on a GE Advantage Workstation version 4.5 (VolumeViewer 9.5) and GE AW Server 3.2 ext3 (VolumeViewer 14 ext4). Organ specific analysis was performed using Organ Level Internal Dose Assessment/EXponential Modeling (OLINDA/EXM) version 1.1 (Vanderbuilt University) with updated weighting factors from the International Commission on Radiation Protection (ICRP) 103 (2007). Individual regions within specific organ sites were taken using 8 cm³ (2×2×2 cm) volumes and SUVmax was recorded at all four time points for the first 19 participants. Detectable radioactivity was seen in the pituitary, lacrimal glands, salivary glands, thyroid, esophagus, stomach, small intestine, large intestine, kidneys, pancreas, bladder, uterus, and prostate gland. Very low/trace uptake was seen in the brain, lungs and liver. Over time, radioactivity was seen to enter into the kidneys with continuous elimination via urine into the bladder. Radioactivity was seen throughout the gastrointestinal tract with anterograde movement over time. The average SUV value for normal lung was 0.56, the range observed was 0.3-1.0 and no significant changes were noted between the imaging times points. Abnormalities that were noted in the lung to date have included, lung carcinoma SUV range 6.0-12.7, lung metastasis range 2.5-5.2 and suspect fibrosis range 2.0-15.4. The mean effective dose for ¹⁸F-αᵥβ₆-BP was 0.02 mSv/MBq.

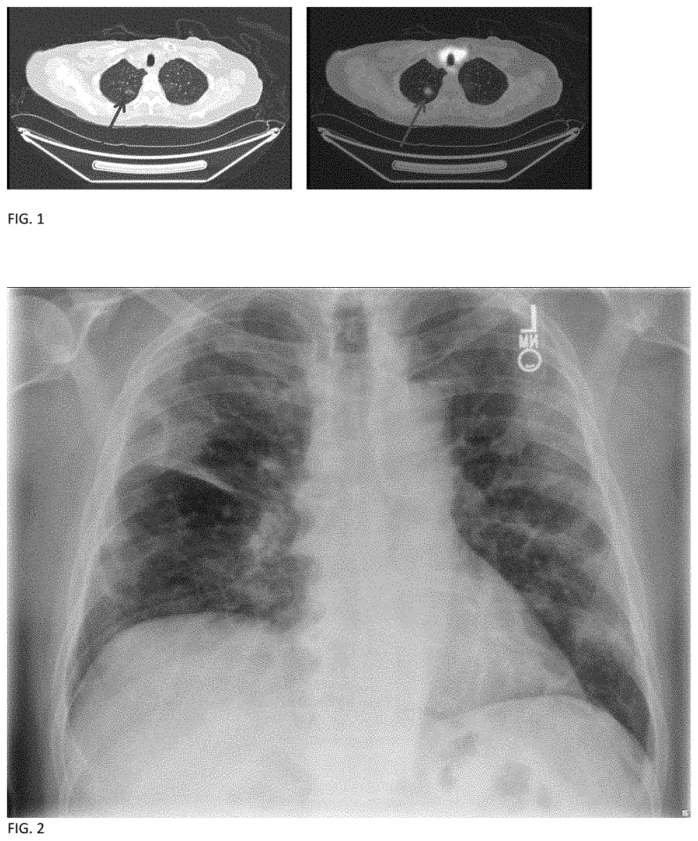

FIG. 1 shows data with imaging using the ¹⁸F-αᵥβ₆-BP in a patient with pancreas cancer. Ground glass opacity in the right upper lobe correlated to an increase in uptake of the ¹⁸F-αᵥβ₆-BP.

### Example 2: Imaging of Lung Tissue of Recovered COVID-19 Patients

The primary objective of this study is to determine the feasibility of ¹⁸F-αᵥβ₆-BP to detect the presence and monitor the regression/progression of lung damage in patients post SARS-CoV-2 infection.

Up to 10 patients with a prior diagnosis of SARS-CoV-2 infection and have since tested negative are recruited to the study. Following a 10 mCi (+20%) intravenous injection (IV) of ¹⁸F-αᵥβ₆-BP, PET/CT images are acquired at 60 minutes. Additional data such as baseline blood samples vital sign (VS) measurements (heart rate, respiratory rate, blood pressure and temperature) are monitored. Region-of-interest analysis (ROI) is performed in the lung. Each participant undergoes up to 3 ¹⁸F-αᵥβ₆-BP PET/CT scans over a 6-month timeframe.

Subjects in the trial include the following characteristics: (i) men and women age >18 yrs.; (ii) Diagnosed with SARS-CoV-2; (iii) 2 sequential COVID negative tests prior to each scan; (iv) no previous lung disease prior to SARS CoV2 infection; (v) lung image (Xray or CT) during infectious/diagnosis period. The subjects actively participate for approximately one (1) day per visit for imaging and up to 3 visits for imaging and participants are followed for up to twelve (12) months for treatment outcomes.

Study endpoints include: Primary Endpoint is the administration of ¹⁸F-αᵥβ₆-BP and PET/CT scans in SARS-CoV-2 patients. Secondary Endpoint is that ¹⁸F-αᵥβ₆-BP demonstrates accumulation in lung damage and correlates with the regression/progression of lung damage over time. Additionally, the correlation of ¹⁸F-αᵥβ₆-BP accumulation in lung to lung damage is assessed.

### Imaging Procedure

Subjects are injected once per imaging session (a maximum of 3 imaging sessions) with up to 10 mCi (+20%) of ¹⁸F-αᵥβ₆-BP as a rapid intravenous bolus (e.g., within 30 secs). Subjects are positioned supine on the scanner table and then undergo scanning from the apex of the skull to the proximal thigh using two minutes per bed position starting at the 1-hour post injection. The PET/CT scan are performed 60 minutes after ¹⁸F-αᵥβ₆-BP injection. Each patient undergoes repeat scanning at 3 months 6 months after the baseline scan to monitor changes.

Depending on the size of the patient sufficient bed positions are used to cover the required range, with an overlap of 11 slices used to minimize axial variations. The time per bed is 2-4 minutes depending on body mass index and time available. Data is reconstructed used the Ordered Subsets—Expectation Maximization (OSEM) algorithm, with 24 subsets and 2 iterations. A 6.4 mm filter is used transaxially and the manufacturer's “standard” filter are used axially. The field of view is 70 cm and the matrix size is 192×192, giving a pixel size of 3.65×3.65 mm.

The PET scans are reviewed in the axial, coronal and sagittal planes using the GE Advantage Windows workstation. Reconstructed PET/CT images are displayed on the GE imaging workstation, reoriented into maximum intensity projection (MIP), transaxial, coronal and sagittal images. PET, fused PET/CT and CT images are reviewed.

The PET images can be interpreted qualitatively and semi-quantitatively on a lesion-by-lesion basis. Semi-quantitative analysis is employed as follows: (a) Regions of interest (ROIs) are placed around tracer avid foci suspicious for lung damage in order to obtain SUV parameters, including SUVₘₐₓ, SUVₚₑₐₖ, SUVₘₑₐₙ and (b) SUV data is recorded along with volumetric and positional information in a standardized form. All SUV measurements are summarized using mean, median, range, and counts where appropriate, and a repeated measures ANOVA model is used to relate the SUVs to the tissue regions. Descriptive statistics for the SUVs are done on a subject basis and a per lesion basis. The sample size is 10 SARS-CoV-2 recovered patients. The primary analytic is to determine whether uptake of ¹⁸F-αᵥβ₆-BP is specifically higher in suspected damaged lung tissue, an SUV mean greater than 2 times normal lung background is considered higher.

¹⁸F-αᵥβ₆-BP Conjugate for Use in the Imaging Protocol

This study uses a microdose study and the actual mass of drug injected based on the 10 mCi (±20%) (molar activity >1Ci/μmole) injection dose of ¹⁸F-αᵥβ₆-BP is less than 50 μgrams (below 100 μgrams). For the maximum injected dose for this study (10 mCi/370 MBq), a participant is exposed to an effective dose of 7.41 mSv.

### Example 3: Human Positron Emission Tomography (PET)/Computed Tomography (CT) Images Using the Integrin αᵥβ₆-Binding Peptide in a Patient Post SARS-CoV-2 Infection

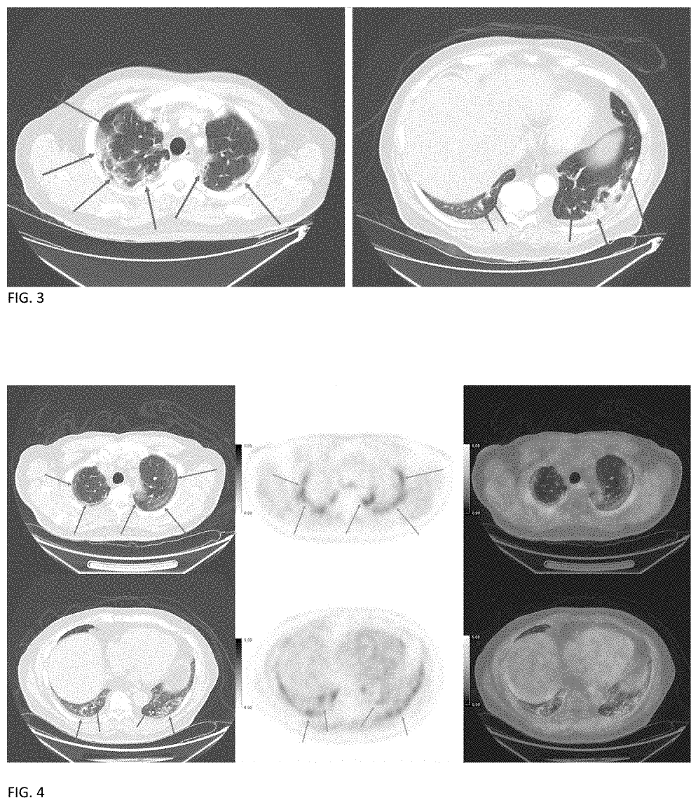

A 71-year-old male with a prior history of hypertension was subjected to the protocol set forth in Example 2. The patient tested negative twice for SARS-CoV-2CoV2 approximately 2 months after initial positive test. The patient's chest X-ray at admission to the hospital showed diffuse pulmonary opacities in the mid and peripheral lungs bilaterally (FIG. 2), consistent with diagnosis of SARS-CoV-2 associated pneumonia. The chest CT scan of the thorax 4 days later showed moderate to severe bilateral central and peripheral patchy areas of ground glass and consolidative changes throughout the lungs (FIG. 3). The ¹⁸F-αᵥβ₆-BP PET/CT images were acquired during recovery 66 days post initial chest CT scan.

The patient was injected with ¹⁸F-αᵥβ₆-BP (340 MBq) as a rapid intravenous bolus. Prior to injection, blood samples were drawn. Immediately before and after the injection the patients vital signs (blood pressure, heart rate, pulse oximetry value and body temperature) were measured. The patient rested for 1 hour prior to the PET/CT scan. The PET scan was performed on a GE Discovery 690 PET/CT scanner at 2 minutes per bed position. A PET/CT acquisition of the thorax with arms up was performed with a typical low dose level CT scan (140 kV ‘smart mA’ [50-350 mA], noise index 20) of the thorax for attenuation correction. Immediately following, a second nonattenuation corrected PET scan was acquired from the skull vertex to the proximal thighs with arms up.

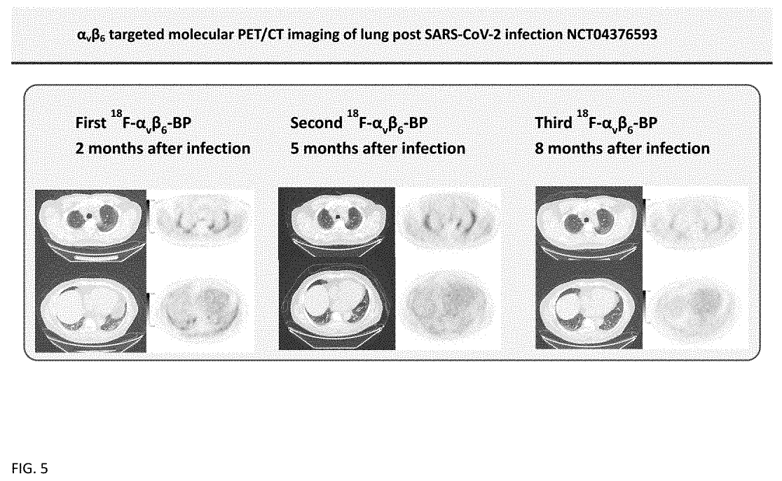

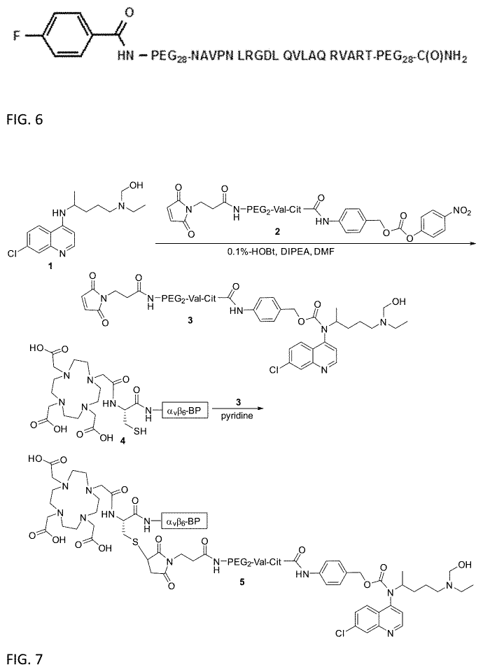

Minimal uptake of ¹⁸F-αᵥβ₆-BP was noted in normal lung parenchyma, with elevated uptake in the lung corresponding to areas of opacities noted on CT. No changes in vital signs were noted during the study and the patient's SpO2 was 100%. The transaxial CT images (FIG. 4 left panels, arrows) through both the upper and lower lungs showed improved areas of bilateral patchy opacities as compared to the initial chest CT (FIG. 3). Transaxial co-registered attenuation corrected ¹⁸F-αᵥβ₆-BP PET images through the upper and lower lungs (scale SUVmax of 5.0, FIG. 4, middle panels) demonstrated elevated uptake of ¹⁸F-αᵥβ₆-BP (SUVmax of 3.0) in areas corresponding to areas of opacities noted on the CT (arrows). Concurrently, regions of normal lung parenchyma by CT demonstrated low levels of ¹⁸F-αᵥβ₆-BP uptake by PET with SUVmax of 0.8-1.0.

This analysis shows the correlation of integrin αᵥβ₆-targeted ¹⁸F-αᵥβ₆-BP PET with SARS-CoV-2 lung damage identified by CT. For areas of lung that corresponded to SARS-CoV-2 related ground glass and consolidation by CT, the SUVmax observed by ¹⁸F-αᵥβ₆-BP PET were approximately 3.0. These values represent an almost 4-fold increase in uptake of ¹⁸F-αᵥβ₆-BP in abnormal vs normal lung tissue and clear visualization of damage.

PET/CT scans were performed at follow-up visits at 3 months and 6 months after the initial scan, using IV administration of 10 mCi ¹⁸F-αᵥβ₆-BP and similar procedures to the initial scan. Results are shown in FIG. 5 with the initial (first) scan shown on the far left, the second scan in the middle and the third scan on the far right of the figure. PET (right panel in each scan) showed elevated activity on PET concordant with abnormalities seen on associated CT with an approximate 3:1 ratio (activity to background lung) on the first and second scans in the upper lobes and reducing to 2:1 on the third scan. The lower lobes on scans 2 and 3 returned to close to background lung activity. CT (left panel of each scan) showed progressive improvement in ground glass and airspace opacities in the upper and to a greater degree the lower lobes of the lungs with resulting small amount of linear arcades vs scarring in the lower lungs on the final scan.

These observations indicate that αᵥβ₆-targeted conjugates such as ¹⁸F-αᵥβ₆-BP can be utilized to detect and monitor the development and progression of lung fibrosis post SARS-CoV-2 infection, and to further follow the tissue remodeling and progression in recovering patients over time.

### Example 4: Human Positron Emission Tomography (PET)/Computed Tomography (CT) Images Using the Integrin αᵥβ₆-Binding Peptide in Patients Post SARS-CoV-2 Infection

The procedures of Example 3 were used to evaluate additional subjects (identified below as PT 1-PT5). CT and PET results are summarized for each subject.

PT 1 underwent 3 scans. CT abnormalities in upper and lower peripheral lungs improved over all three scans with mild residual atelectasis/scarring on final scan. ¹⁸F-αᵥβ₆-BP PET activity was seen well above background lung (3×) which persisted but diminished over the three scans with resolution of activity in the lower lungs.

PT 2 underwent 3 scans. CT scans showed mild abnormalities in the upper and lower lungs that effectively resolved by scan 3. ¹⁸F-αᵥβ₆-BP PET activity was seen predominantly in the right upper lobe (>2× background) and improved to resolution by scan 3.

PT 3 underwent 3 scans. No significant abnormality was observed on CT for all three scans. ¹⁸F-αᵥβ₆-BP PET activity was seen within the central (and pulmonary) vasculature on all three exams. Pulmonary activity was more prominent in the dependent areas and suspected to be vascular in nature.

PT 4 underwent 2 scans. CT scans showed extensive peripheral hazy densities along the mid and lower lungs with significant improvement on scan 2. ¹⁸F-αᵥβ₆-BP PET activity aligned with areas of density (˜3×+above background) that improved to ˜2×+by scan 2.

PT 5 underwent 1 scan. No abnormalities were observed on CT of lungs. ¹⁸F-αᵥβ₆-BP PET showed no elevated activity above background.

### Example 5: PET/CT Imaging Using the Integrin ᵥP6 Binding Peptide to Monitor “Long-Hauler” SARS-CoV-2 Infection

The procedures of Example 3 were utilized to assess the progress of subjects categorized as “long haulers” or having “long COVID.” Subjects are monitored with ¹⁸F-αᵥβ₆-BP PET imaging, and optionally CT scans. Subjects are assessed post-infection after such subjects test negative for the presence of the virus, but continue to experience one or more symptoms of long COVID such as shortness of breath, a need for continued oxygen treatment, coughing, ongoing fatigue, body aches, joint pain, loss of taste and smell, difficulty sleeping, headaches or brain fog. Subjects are imaged on a periodic basis such as every 1 month or every 2 months or every 3 months or every 6 months to assess for lung damage and any changes (e.g., improvements or further damage over time). In some instances, such subjects are receiving one or more COVID treatments such as anti-viral drugs and/or COVID-directed antibody treatments during the course of the imaging and the effects of the treatment are assessed.

### Example 6: Targeted Therapeutics for Treatment of SARS-CoV-2 Infection

To deliver a therapeutic agent for SARS-CoV-2 infection, an αᵥβ₆-targeting peptide (such as A20FMDV that was used, for example, in Examples 1-3) is conjugated to a therapeutic agent, such as hydroxychloroquine (HCQ), through a cleavable linker. Such conjugate is designed to deliver HCQ specifically to diseased (virus-infected) epithelial cells, and once inside the cells, the HCQ is released to function and stop viral replication. An exemplary synthesis in shown in FIG. 7.

Without being bound by any particular theory, the conjugate selectively binds to injured (virus-infected) lung epithelial cells and enters the cell through endocytosis. Once inside the endosomes, the linkage between HCQ and the αᵥβ₆-targeting peptide (e.g., A20FMDV) is broken due to the protease activity and low pH of endosomes, and the released HCQ prevents and inhibits viral replication (as free HCQ). Compared with free HCQ, which enters both healthy and virus infected cells, the conjugate (e.g., HCQ-A20FMDV conjugate) is targeted more specifically to injured (virus-infected) cells, and therefore has higher therapeutic efficacy and lower toxicity.

### Methodology

The HCQ-A20FMDV conjugate is synthesized as shown in FIG. 7, using the A20FMDV peptide containing a PEG at the N-terminus and C-terminus of the peptide. Bi-PEGylation was performed as set forth in U.S. Pat. No. 10,919,932, which is incorporated by reference herein in its entirety.

Hydroxychloroquine (1) is attached to a cathepsin B cleavable linker, which can also hydrolyze from endosomal enzymes and pH via reaction with the carbonate-dipeptide (Val-Cit) maleimide linker 2 using catalytic hydroxybenzotriazole (HOBt) to yield 3. The DOTA-C-αᵥβ₆-BP construct 4 is built on solid support using Fmoc chemistry, cleaved, and purified, after which it is conjugated to 3 through a 1,4-conjugate addition reaction to produce peptide drug conjugate (PDC) 5.

The binding specificity of HCQ-A20FMDV (5) is characterized with competitive ELISA against biotinylated natural ligand-LAP v06 and αᵥβ₆. Radioactivity labeling: [⁶⁴Cu]C₂ in 0.5 M HCl is diluted in 0.1M NH₄OAc, pH 6 and incubated with the conjugate 5 at 40° C. for 1 hr. The crude reaction products are purified with HPLC.

The serum stability is tested with ⁶⁴Cu-5 in mouse and human sera as set forth in Tang, Y. C., et al., Identification, Characterization, and Optimization of Integrin alpha(v)beta(6)-Targeting Peptides from a One-Bead One-Compound (OBOC) Library: Towards the Development of Positron Emission Tomography (PET) Imaging Agents. Molecules, 2019. 24(2): p. 16. In brief, formulated ⁶⁴Cu-5 (˜100 μCi in PBS) is incubated with mouse serum (500 μL) at 37° C. for 1 h. An aliquot of ⁶⁴Cu-5 in mouse serum (100 μL) is taken at 1 h, mixed with absolute ethanol (500 μL, 4° C.), and centrifuged to precipitate serum protein. The supernatant is diluted with HPLC solvent A before radio-HPLC analysis to determine the fraction of intact ⁶⁴Cu-5.

Cell binding specificity is tested with DX3puroβ6 [αᵥβ₆(+)] and DX3puro [αᵥβ₆(−)]cells, similar to Tang et al. In brief, 0.2 μCi of ⁶⁴Cu-5 is added to 3.75×10⁶ cells and incubated for 1 h at 37° C. (n=4/cell line). Following centrifugation, supernatant is removed, and the cell pellet is washed with 0.5 mL SF-DMEM. The fraction of bound radioactively is determined by with a γ-counter. To determine the fraction of internalized radioactivity, the cells are subsequently treated with acidic wash buffer to release surface-bound activity and measured in a γ-counter.

The cell toxicity is tested with Vero cells (ATCC® CCL-81™) using WST-1 assay. Vero cells are monkey kidney epithelial cells, and are reported able to be infected by SARS-CoV-2. Cells are seeded into 96-well plates at a density of 5 k cells/well and grown for 24 hours prior to treatment, then the cells are incubated with media containing either free HCQ or HCQ-A20FMDV at 10⁻⁵, 10⁻⁴, 10⁻³, 10⁻², 10⁻¹, 1, 10, and 100 μM for 24 or 48 hours (n=3/concentration/time point), after which the cell viability is evaluated with WST-1 assay.

In vitro anti-SARS-CoV-2 activity of HCQ-A20FMDV is tested with Vero cells (ATCC® CCL-81™), similar to Yao, X., et al., In Vitro Antiviral Activity and Projection of Optimized Dosing Design of Hydroxychloroquine for the Treatment of Severe Acute Respiratory Syndrome coronavirus 2 (SARS-CoV-2). Clinical infectious diseases: an official publication of the Infectious Diseases Society of America, 2020. Such test is conducted at a BSL-3 lab. In brief, cells are seeded into 96-well plates at a density of 1×10⁴ cells/well and grown for 24 hours prior to infection and treatment. Cells are infected at a multiplicity of infection (MOI) of 0.01 (100 PFU/well) for 2 hours at a temperature of 37° C. Virus input is washed with DMEM and the cells are then treated with medium containing either HCQ or HCQ-A20FMDV at 10⁻⁵, 10⁻⁴, 10⁻³, 10⁻², 10⁻¹, 1, 10, and 100 μM for 24 or 48 hours (n=3/concentration/time point). The supernatant is collected and the RNA is extracted and analyzed by relative quantification using RT-PCR e.g., using methods such as in Huang, C., et al., Clinical features of patients infected with 2019 novel coronavirus in Wuhan, China. Lancet, 2020. 395(10223): p. 497-506.

The maximum tolerated dose (MTD) is tested in mouse with free HCQ and HCQ-A20FMDV similarly to Aston, W. J., et al., A systematic investigation of the maximum tolerated dose of cytotoxic chemotherapy with and without supportive care in mice. Bmc Cancer, 2017. 17: p. 10. Two endpoints, weight loss >15% or clinical score >2, are used. Clinical signs are scored by observing activity, appearance and body condition: 0, normal; 1 slight deviation from normal; and 2, moderate deviation from normal. The safety of free HCQ and HCA-A20FMDV is tested with doses of equivalent to 9, 12, 15, 18, 21 mg/kg HCQ (n=4 for each dose). When either of these endpoints is met, the prior dose is set as the MTD.

Cynomolgus macaques have been reported to be a non-human primate model of COVID-19 and are used to test the therapeutic efficacy of HCQ-A20FMDV compared with HCQ. Healthy, adult female cynomolgus macaques (Macaca fascicularis) are handled in an ABSL3 biocontainment laboratory. Sixteen female cynomolgus macaques (ages 5-20 years) weighing between 3.5 and 5.0 kg are distributed evenly regarding to age over four groups of four animals. Each group consists of two young adult animals (5 years) and two aged animals (15-20 years).

| Group | A | B | C | D |
|---|---|---|---|---|
| Treatment | HCQ | HCQ | HCQ-A20FMDV | HCQ-A20FMDV |
| Euthanization | 6 | 21 | 6 | 21 |
| day, p.i. | | | | |

Animals are inoculated with SARS-CoV-2 under anesthesia via a combination of intratracheal (4.5 ml) and intranasal (0.25 ml per nostril) routes with a suspension containing 2×10⁵ TCID50 per ml PBS (total infectious dose=10⁶ TCID50). Animals are treated with free HCQ or HCQ-A20FMDV (equivalent to 1 mg/kg HCQ) on Day 2 p.i. while i.v. injection. Animals are anesthetized for challenge, blood collection, treatment, and swabs of nasal, throat and rectal mucosa on days 0, 1, 2, 3, 4, 6, 8, 10, 14, 18 and 21 p.i. After euthanization, the animals are autopsied on day 4 p.i. with collection of tissue specimens from respiratory, digestive urinary, and cardiovascular tracts, endocrine and central nervous systems, as well as various lymphoid organs.

### EXEMPLARY EMBODIMENTS

Exemplary embodiments provided in accordance with the presently disclosed subject matter include, but are not limited to, the claims and the following embodiments:
1. A method of imaging virus-related lung damage comprising:
   (a) administering to a subject a conjugate comprising an αᵥβ₆-binding peptide covalently attached to an imaging agent, wherein the subject has been exposed to or is suspected of exposure to a respiratory virus causative of lung damage; and
   (b) detecting the conjugate in lung tissue of the subject to determine the location and/or concentration of the conjugate in the lung tissue, thereby imaging the lung damage.
2. The method according to embodiment 1, wherein the respiratory virus is a severe acute respiratory syndrome (SARS) coronavirus.
3. The method according to embodiment 2, wherein the SARS coronavirus is selected from the group consisting of SARS-CoV, SARS-CoV-2, and variants thereof.
4. The method according to any one of embodiments 1-3, wherein the subject has been diagnosed as a COVID-19 patient.
5. The method according to any one of embodiments 1-3, wherein the subject has been previously quarantined for COVID-19 exposure.
6. The method according to any one of embodiments 1-3, wherein the subject has been infected with SARS-CoV-2 or a variant thereof.
7. The method according to embodiment 6, wherein the imaging is performed about 1 day, about 2 days, about 3 days, about 4 days, about 5 days, about 6 days, about 7 days, about 8 days, about 9 days, about 10 days, about 11, days, about 12 days, about 13 days, about 14 days, about 15 days, about 16 days, about 17 days, about 18 days, about 19 days, about 20 days, about 21 days, or more than about 21 days after infection with SARS-CoV-2 or a variant thereof.
8. The method according to any one of embodiments 1-7, wherein the subject is symptomatic for COVID-19.
9. The method according to any one of embodiments 1-7, wherein the subject is asymptomatic for COVID-19.
10. The method according to any one of embodiments 1-7, wherein the subject has recovered from COVID-19.
11. The method according to embodiment 10, wherein the imaging is performed about 1 day, about 2 days, about 3 days, about 4 days, about 5 days, about 6 days, about 7 days, about 8 days, about 9 days, about 10 days, about 11, days, about 12 days, about 13 days, about 14 days, about 15 days, about 16 days, about 17 days, about 18 days, about 19 days, about 20 days, about 21 days, or more than about 21 days after the subject has recovered from COVID-19.
12. The method according to any one of embodiments 1-7, wherein the subject is diagnosed as a COVID long-hauler.
13. The method according to any one of embodiments 1-12, wherein the imaging is performed at a repeating interval.
14. The method according to embodiment 13, wherein the interval is about 1 week, about 2 weeks, about 3 weeks, about 4 weeks, about 5 weeks, about 6 weeks, about 7 weeks, about 8 weeks, about 9 weeks, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 2 years, about 3 years, about 4 years, about 5 years, or longer.
15. The method according to any one of embodiments 1-14, wherein the lung damage comprises pulmonary fibrosis.
16. The method according to any one of embodiments 1-15, wherein steps (a) and (b) are repeated during the course of infection with the respiratory virus.
17. The method according to any one of embodiments 1-15, wherein steps (a) and (b) are repeated during the course of recovery from infection with the respiratory virus.
18. The method according to any one of embodiments 1-17, wherein the imaging agent is selected from the group consisting of a radionuclide, biotin, a fluorophore, a fluorescent protein, an antibody, an enzyme, and combinations thereof.
19. The method according to embodiment 18, wherein the imaging agent is a radionuclide selected from the group consisting of ¹¹C, ¹³N, ¹⁵O, ¹⁸F, ¹⁹F, ⁶¹Cu, ⁶²Cu, ⁶⁴Cu, ⁶⁷Cu, ⁶⁸Ga, ¹¹¹In, ¹²⁴I, ¹²⁵I, and ¹³¹I.
20. The method according to any one of embodiments 1-19, wherein the conjugate is detected by Magnetic Resonance Imaging (MRI), Magnetic Resonance Spectroscopy (MRS), Single Photon Emission Computerized Tomography (SPECT), Positron Emission Tomography (PET), or optical imaging.
21. The method according to any one of embodiments 1-20, wherein the conjugate is administered to the subject by intravenous injection.
22. The method according to any one of embodiments 1-21, wherein step (b) is performed between about 10 minutes and about 180 minutes after step (a).
23. The method according to any one of embodiments 1-21, wherein step (b) is performed at about 15 minutes, about 30 minutes, about 60 minutes, about 75 minutes, about 90 minutes, or about 120 minutes after step (a).
24. The method according to any one of embodiments 1-23, further comprising repeating steps (a) and (b) at one or more additional time points and determining the extent of the regression or progression of lung damage over time.
25. The method according to any one of embodiments 1-24, wherein the amount or concentration of the conjugate is correlative to the severity of lung damage caused by the respiratory virus.
26. A method of treating virus-related lung damage comprising: administering to a subject a conjugate comprising an αᵥβ₆-binding peptide covalently attached to a therapeutic agent, wherein the subject has been exposed, is diagnosed with or suffers from a respiratory virus causative of lung damage, thereby treating the lung damage.
27. The method according to embodiment 26, wherein the respiratory virus is a severe acute respiratory syndrome (SARS) coronavirus.
28. The method according to embodiment 27, wherein the SARS coronavirus is selected from the group consisting of SARS-CoV, SARS-CoV-2, and variants thereof.
29. The method according to any one of embodiments 26-28, wherein the subject has been diagnosed as a COVID-19 patient.
30. The method according to any one of embodiments 26-28, wherein the subject has been infected with SARS-CoV-2 or a variant thereof.
31. The method according to any one of embodiments 26-28, wherein the subject is symptomatic for COVID-19.
32. The method according to any one of embodiments 26-28, wherein the subject is diagnosed as a COVID long-hauler.
33. The method according to any one of embodiments 26-32, wherein the conjugate is administered to the subject by intravenous injection.
34. The method according to any one of embodiments 26-33, wherein the lung damage comprises pulmonary fibrosis.
35. The method according to any one of embodiments 26-34, wherein the therapeutic agent is an anti-inflammatory, an anti-fibrotic, or an antiviral agent.
36. The method according to any one of embodiments 26-35, wherein the therapeutic agent is a small molecule.
37. The method according to any one of embodiments 26-35, wherein the therapeutic agent is selected from the group consisting of nintedanib, pirfenidone, remdesivir, and hydroxychloroquine.
38. The method according to any one of embodiments 26-37, wherein the therapeutic agent is covalently attached to the peptide via a linker.
39. The method according to embodiment 38, wherein the linker is a cleavable linker.
40. The method according to any one of embodiments 1-25, wherein the conjugate further comprises a polyethylene glycol (PEG) moiety covalently attached to the amino-terminus of the peptide or a PEG moiety covalently attached to the C-terminus of the peptide.
41. The method according to embodiment 40, wherein the imaging agent is covalently attached to the peptide or the PEG moiety.
42. The method according to any one of embodiments 26-39, wherein the conjugate further comprises a PEG moiety covalently attached to the amino-terminus of the peptide or a PEG moiety covalently attached to the C-terminus of the peptide.
43. The method according to embodiment 42, wherein the therapeutic agent is covalently attached to the peptide or the PEG moiety.
44. The method according to any one of embodiments 40-43, wherein the PEG moiety has a molecular weight of less than about 3000 daltons (Da).
45. The method according to any one of embodiments 40-44, wherein the PEG moiety is selected from the group consisting of PEG₁₂ (PEG 800), PEG₂₈ (PEG 1500), and (PEG₂₈)₂ (PEG 1500×2).
46. The method according to any one of embodiments 1-25, wherein the conjugate further comprises a first PEG moiety covalently attached to the amino-terminus of the peptide and a second PEG moiety covalently attached to the C-terminus of the peptide.
47. The method according to embodiment 46, wherein the imaging agent is covalently attached to the peptide, the first PEG moiety, or the second PEG moiety.
48. The method according to any one of embodiments 26-39, wherein the conjugate further comprises a first PEG moiety covalently attached to the amino-terminus of the peptide and a second PEG moiety covalently attached to the C-terminus of the peptide.
49. The method according to embodiment 48, wherein the therapeutic agent is covalently attached to the peptide, the first PEG moiety, or the second PEG moiety.
50. The method according to any one of embodiments 46-49, wherein the first PEG moiety and/or the second PEG moiety each have a molecular weight of less than about 3000 Da.
51. The method according to any one of embodiments 46-50, wherein the first PEG moiety and/or the second PEG moiety are independently selected from the group consisting of PEG₁₂ (PEG 800), PEG₂₈ (PEG 1500), and (PEG₂₈)₂ (PEG 1500×2).
52. The method according to any one of embodiments 1-51, wherein the αᵥβ₆-binding peptide comprises an RGD sequence.
53. The method according to embodiment 52, wherein the αᵥβ₆-binding peptide comprises the amino acid sequence RGDLX₁X₂X₃, and wherein X₁ and X₂ are independently selected amino acids and X₃ is L or I.
54. The method according to embodiment 53, wherein the αᵥβ₆-binding peptide comprises the amino acid sequence RGDLX₁X₂X₃AQX₆, wherein X₆ is R or K.
55. The method according to embodiment 52 or 53, wherein the αᵥβ₆-binding peptide comprises an amino acid sequence selected from the group consisting of NAVPNLRGDLQVLAQRVART (A20FMDV2 K16R), NAVPNLRGDLQVLAQKVART (A20FMDV2), and GNGVPNLRGDLQVLGQRVGRT.
56. The method according to any one of embodiments 1-25, wherein the conjugate comprises the structure:

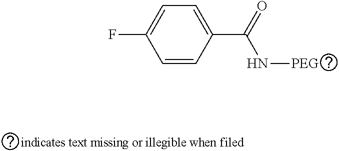

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications mentioned in this specification are herein incorporated by reference to the same extent as if each individual publication, patent, or patent application was specifically and individually indicated to be incorporated by reference.

1. A method of imaging virus-related lung damage comprising:
(a) administering to a subject a conjugate comprising an αᵥβ₆-binding peptide covalently attached to an imaging agent, wherein the subject has been exposed to or is suspected of exposure to a respiratory virus causative of lung damage; and
(b) detecting the conjugate in lung tissue of the subject to determine the location and/or concentration of the conjugate in the lung tissue, thereby imaging the lung damage.

2. The method according to claim 1, wherein the respiratory virus is a severe acute respiratory syndrome (SARS) coronavirus.

3. The method according to claim 2, wherein the SARS coronavirus is selected from the group consisting of SARS-CoV, SARS-CoV-2, and variants thereof.

4-6. (canceled)

7. The method according to claim 1, wherein the imaging is performed about 1 day, about 2 days, about 3 days, about 4 days, about 5 days, about 6 days, about 7 days, about 8 days, about 9 days, about 10 days, about 11, days, about 12 days, about 13 days, about 14 days, about 15 days, about 16 days, about 17 days, about 18 days, about 19 days, about 20 days, about 21 days, or more than about 21 days after infection with SARS-CoV-2 or a variant thereof.

8. The method according to claim 1, wherein the subject is symptomatic for COVID-19.

9. The method according to claim 1, wherein the subject is asymptomatic for COVID-19.

10. (canceled)

11. (canceled)

12. The method according to claim 1, wherein the subject is diagnosed as a COVID long-hauler.

13. The method according to claim 1, wherein the imaging is performed at a repeating interval.

14. The method according to claim 13, wherein the interval is about 1 week, about 2 weeks, about 3 weeks, about 4 weeks, about 5 weeks, about 6 weeks, about 7 weeks, about 8 weeks, about 9 weeks, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 2 years, about 3 years, about 4 years, about 5 years, or longer.

15. The method according to claim 1, wherein the lung damage comprises pulmonary fibrosis.

16. (canceled)

17. (canceled)

18. The method according to claim 1, wherein the imaging agent is selected from the group consisting of a radionuclide, biotin, a fluorophore, a fluorescent protein, an antibody, an enzyme, and combinations thereof.

19. The method according to claim 18, wherein the imaging agent is a radionuclide selected from the group consisting of ¹¹C, ¹³N, ¹⁵O, ¹⁸F, ¹⁹F, ⁶¹Cu, ⁶²Cu, ⁶⁴Cu, ⁶⁷Cu, ⁶⁸Ga, ¹¹¹In, ¹²⁴I, ¹²⁵I and ¹³¹I.

20-25. (canceled)

26. A method of treating virus-related lung damage comprising:
administering to a subject a conjugate comprising an αᵥβ₆-binding peptide covalently attached to a therapeutic agent, wherein the subject has been exposed, is diagnosed with or suffers from a respiratory virus causative of lung damage, thereby treating the lung damage.

27. The method according to claim 26, wherein the respiratory virus is a severe acute respiratory syndrome (SARS) coronavirus.

28-33. (canceled)

34. The method according to claim 26, wherein the lung damage comprises pulmonary fibrosis.

35. The method according to claim 26, wherein the therapeutic agent is an anti-inflammatory, an anti-fibrotic, or an antiviral agent.

36-39. (canceled)

40. The method according to claim 1, wherein the conjugate further comprises a polyethylene glycol (PEG) moiety covalently attached to the amino-terminus of the peptide or a PEG moiety covalently attached to the C-terminus of the peptide or a combination thereof.

41-51. (canceled)

52. The method according to claim 1, wherein the αᵥβ₆-binding peptide comprises an RGD sequence.

53. The method according to claim 52, wherein: (a) the αᵥβ₆-binding peptide comprises the amino acid sequence RGDLX₁X₂X₃: or (b) the αᵥβ₆-binding peptide comprises the amino acid sequence RGDLX₁X₂X₃AQX₆, wherein X₁ and X₂ are independently selected amino acids, X₃ is L or I, and X₆ is R or K.

54. (canceled)

55. The method according to claim 53, wherein the αᵥβ₆-binding peptide comprises an amino acid sequence selected from the group consisting of NAVPNLRGDLQVLAQRVART (A20FMDV2 K16R), NAVPNLRGDLQVLAQKVART (A20FMDV2), and GNGVPNLRGDLQVLGQRVGRT.

56. The method according to claim 1, wherein the conjugate comprises the structure:

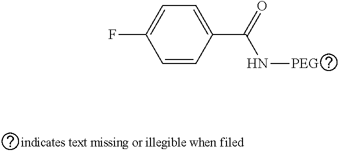

57. The method according to claim 26, wherein the αᵥβ₆-binding peptide comprises an RGD sequence.

58. The method according to claim 57, wherein: (a) the αᵥβ₆-binding peptide comprises the amino acid sequence RGDLX₁X₂X₃; or (b) the αᵥβ₆-binding peptide comprises the amino acid sequence RGDLX₁X₂X₃AQX₆, wherein X₁ and X₂ are independently selected amino acids, X₃ is L or I, and X₆ is R or K.

59. The method according to claim 58, wherein the αᵥβ₆-binding peptide comprises an amino acid sequence selected from the group consisting of NAVPNLRGDLQVLAQRVART (A20FMDV2 K16R), NAVPNLRGDLQVLAQKVART (A20FMDV2), and GNGVPNLRGDLQVLGQRVGRT.
